(19) United States
(12) Patent Application Publication
(10) Pub. No.: US 2026/0335459 A1
(43) Pub. Date: Oct. 29, 2026

(54) BI-SPECIFIC AGENT FOR TARGETED PLASMA CELL-DEPLETION AND TREATMENT OF ANTI-VIRAL IMMUNODEFICIENCY

(71) Applicant: DEUTSCHES RHEUMA-FORSCHUNGSZENTRUM BERLIN, Berlin (DE)

(72) Inventors: Peter K. JANI, Berlin (DE); Andreas RADBRUCH, Fresdorf (DE); Falk HIEPE, Berlin (DE); Qingyu CHENG, Berlin (DE)

(73) Assignee: DEUTSCHES RHEUMA-FORSCHUNGSZENTRUM BERLIN, Berlin (DE)

(21) Appl. No.: 19/114,177

(22) PCT Filed: Sep. 22, 2023
(86) PCT No.: PCT/EP2023/076248

§ 371 Date: Mar. 21, 2025

(30) Foreign Application Priority Data
Sep. 23, 2022 (EP) 22197395.1

(51) Int. Cl. A61K 47/68; A61P 37/04; G01N 33/564

(52) U.S. Cl. CPC A61K 47/6813; A61K 47/6849; A61P 37/04; G01N 33/564; G01N 2333/56

(57) ABSTRACT

The invention relates to a bi-specific agent, comprising an anchor region capable of binding a plasma cell, and a binding region associated with the anchor region, wherein the binding region comprises an epitope of an interferon, an interferon receptor or combination thereof, bound by one or more autoantibodies. The invention further relates to the used of said a bi-specific agent, in the treatment of an anti-viral immunodeficiency in a patient, wherein the anti-viral immunodeficiency in the patient is associated with the presence of one or more autoantibodies that bind an epitope of an interferon, an interferon receptor or combination thereof. The invention also relates to an in vitro assay for identifying autoantibodies that bind an epitope of an interferon, an interferon receptor or combination thereof and to kits for performing said assay.

The invention lies in the field of molecular biology and medicine, more specifically in the field of immunobiology, infection medicine and the treatment of immunodeficiency.

The invention relates to a bi-specific agent, comprising an anchor region capable of binding a plasma cell, and a binding region associated with the anchor region, wherein the binding region comprises an epitope of an interferon, an interferon receptor or combination thereof, capable of binding one or more autoantibodies. The invention further relates to the use of said a bi-specific agent in the treatment of an anti-viral immunodeficiency in a patient, wherein the anti-viral immunodeficiency in the patient is associated with the presence of one or more autoantibodies that bind an epitope of an interferon, an interferon receptor or combination thereof. The invention also relates to an in vitro assay for identifying autoantibodies that bind an epitope of an interferon, an interferon receptor or combination thereof and to kits for performing said assay. The invention further relates to means and methods for providing a bi-specific agent of the invention in a personalized manner for a patient, wherein the epitope (of an interferon, an interferon receptor or combination thereof) of the binding region, is an epitope that is capable of binding, or is bound by an autoantibody present in the patient.

### BACKGROUND OF THE INVENTION

An autoimmune condition leading to (in some cases severe) adaptive immunodeficiency in combating or preventing viral infection is prevalent among the elderly. Attention has been drawn towards this antiviral immunodeficiency of patients being 60 years of age or older especially during the COVID-19 pandemic. Recent studies show that 4% of people over the age of 70 develop autoantibodies against components of the immune system, such as interferons or interferon receptors. Especially in the case of interferons or interferon receptors, such autoantibodies can significantly impair the immune defense of patients against viruses, as interferon signaling constitutes the prime alarm signal for the immune system in case of viral infections. This deficiency predisposes elderly patients to severe disease and death by viral infections, e.g., COVID-19 (Bastard P, et al.; Science. 2020).

Efficient strategies for stabilizing and restoring anti-viral immunity in elderly patients are still lacking to date and no clinical treatment guideline is available for patients with severe viral infection having high anti-interferon autoantibody titers.

Recombinant interferon administration itself has no curative potential and would be required to be administrated continuously to subjects over the age of 60 for the rest of their life.

The current approach towards improving the immunocompetence of the elderly against viral infections are vaccinations. However, vaccinations only protect against specific types or even only subtypes of viruses, wherefore potential future viral infections of a patient have to be estimated in advance to prevent potential future infections that might comprise a serious course of disease or even lead to the death of a patient. In addition, the protective immune response of elderly patients to vaccinations dramatically decreases with increasing age.

Therefore, there exists a high unmet need to support and restore general anti-viral immunity in elderly subjects, which suffer from anti-viral immunodeficiency caused by autoantibodies against components of the immune system.

### SUMMARY OF THE INVENTION

The inventors surprisingly found that targeting plasma cells producing anti-interferon signaling autoantibodies results in the selective ablation of autoantibodies targeting interferon-signaling, and is capable of regenerating, improving and/or restoring immunocompetence of affected patients, in particular in elderly patients. Thereby the humoral immunity that patients acquired during their lifetime can be preserved or restored, which significantly contributes to their healthy aging.

In light of the prior art, one objective of the invention is to provide improved or alternative means for improving immunocompetence against viral infection, especially in elderly subjects. Another objective is the provision of means that improve antigen-unspecific immune responses and reduce risk of adverse events caused by viral infections.

This problem is solved by the features of the independent claims. Preferred embodiments of the present invention are provided by the dependent claims.

The invention therefore relates to a bi-specific agent, comprising: an anchor region capable of binding a plasma cell, and a binding region associated with the anchor region, wherein the binding region comprises an epitope of an interferon, an interferon receptor or combination thereof, capable of binding one or more autoantibodies.

It was entirely surprising that the invention facilitates the determination of the presence of interferon or interferon receptor specific autoantibodies in a patient on one hand, while also providing specific means for targeting such autoantibodies and/or the antibody producing plasma cells in each patient individually. Aspects of the invention facilitate the determination of the binding epitopes on interferons and/or interferon receptors of autoantibodies present in an individual patient und further enable the treatment of the immunodeficiency induced in said patient by said autoantibodies.

In embodiments, the binding region, comprising an epitope of an interferon, an interferon receptor or combination thereof, is capable of binding one or more autoantibodies. In embodiments, the binding region, comprising an epitope of an interferon, an interferon receptor or combination thereof, is bound by one or more autoantibodies after administration to a patient. In embodiments, the binding region, comprising an epitope of an interferon, an interferon receptor or combination thereof, is a known target (autoantigen) of one or more autoantibodies present in a subject with an immunodeficiency. In embodiments, the binding region, comprising an epitope of an interferon, an interferon receptor or combination thereof, is selected after determination of anti-interferon or anti-interferon receptor autoantibodies in the patient. As used herein, the term “capable of binding one or more autoantibodies” may be used interchangeably with “bound by one or more autoantibodies”. The term “bound by one or more autoantibodies” does not necessarily refer to an agent in which an autoantibody is bound prior to administration, rather to an autoantigen (autoepitope) that would be bound by a pathogenic anti-interferon or anti-interferon receptor autoantibody in a patient.

For determining the specific epitopes of the autoantibodies, in vitro assays are provided herein that facilitate the identification of the specific interferon or interferon receptor epitopes of autoantibodies in a patient sample. Once these specific target epitopes of a patient's autoantibodies are known, the intervention provides means for the provision of interferon and/or interferon receptor epitope-comprising bi-specific agents (e.g., affinity matrices) that facilitate the specific depletion of plasma cells secreting interferon and/or interferon receptor epitope-specific autoantibodies in a patient. The treatment options provided herein are able to regenerate, restore, improve and/or preserve the humoral immunity in individual patients to improve immunity against viral and/or intracellular pathogen infections.

Although addressing viral infection is a preferred embodiment of the invention, any pathogen infection in a subject may be worsened by a weakened immune response caused by anti-interferon or anti-interferon receptor autoantibodies. Thus, the approach described in the present invention is applicable towards treating and/or preventing any given pathogen infection in a subject, wherein viral infections represent a preferred embodiment. The relevant medical condition for treatment is thus any disorder in which an IFN response plays a role, for example any infection to which an IFN-mediated immune response provides a beneficial (anti-infective) effect.

The means presented herein also provide the potential for routine testing of individuals above the age of 60 for the presence of interferon and/or interferon receptor specific autoantibodies, in order to test susceptibility to severe courses of viral infections. The present invention enables the mapping of the epitope specificities of autoantibodies detected in patient samples by providing in vitro assays suitable for the use, for example, in binding-inhibition assays or peptide-arrays. The present invention provides means enabling the personalized treatment of patients for the epitope-specific depletion of plasma cells secreting/producing autoantibodies in a patient in order to prevent severe viral or intracellular pathogen infections.

In embodiments of the bi-specific agent according to the invention, the autoantibody that binds the epitope of an interferon, an interferon receptor or a combination thereof, is presented on and/or released from a plasma cell.

In embodiments of the bi-specific agent according to the invention, the anchor region comprises a monoclonal antibody or antigen-binding fragment thereof that binds a plasma cell, preferably the monoclonal antibody or antigen-binding fragment specifically binds a plasma cell marker presented on the surface of a plasma cell.

In embodiments, an anchor region is capable of binding to an antigen on the surface of a plasma cell. In one aspect the affinity constants for the interaction of the anchor region with a cell surface antigen, may be in the range of 10⁶ to 10⁹ M⁻¹, or may be greater than 10⁶, greater than 10⁷, greater than 10⁸ or greater than 10⁹ M⁻¹.

In embodiments, an anchor region capable of binding to a plasma cell means that the anchor region will preferentially bind to plasma cells with reduced or no or minimal binding in comparison to another cell type, e.g., B and T lymphocytes. Minimal binding might be less than 50%, e.g., less than 40%, less than 30%, less than 20% or less than 10% of the binding to other cell types, e.g., B cells or T cells, as assessed by, e.g., association or dissociation constants. Suitably specific binding can be achieved by binding to molecules that are mainly or uniquely found on the surface of the plasma cell, or are found at a higher number on plasma cells than on other cell types, e.g., than B cell or T cells.

In embodiments, an anchor region binds to a plasma cell with at least 2-fold greater affinity, such as at least 5 fold greater, 10 fold greater, 50 fold greater, 100 fold greater, or more affinity, with respect to a non-plasma cell, such as a B or T cell. In one aspect the anchor region binds to a plasma cell in at least a 2-fold greater number, such as at least 5-fold greater, 10-fold greater, 50-fold greater, 100-fold greater number, or more, than it binds to a non-plasma cell, such as a B or T cell.

In embodiments, the binding of an anchor region to a plasma cell can be distinguished from binding of the same anchor region to another (non-plasma cell), by analysis of cell killing commonly known in the art or as described herein, wherein suitably specific binding of the anchor region gives an increase in cell killing of plasma cells vs. non-plasma cells, such as B or T cells, generally in the order of 2-fold, 3-fold, 4-fold, 5-fold, 10-fold, 50-fold, 100-fold or even more. In this way the cell killing as described herein is preferential for plasma cells.

In embodiments of the bi-specific agent according to the invention the anchor region is capable of specifically binding to plasma cell surface antigens selected from the group comprising CD138, CD38, BCMA, TACI, CD98, SLAM7 (CD319), CD27 or any combination thereof.

In embodiments of the bi-specific agent according to the invention the anchor region is capable of specifically binding to plasma cell surface antigens selected from the group comprising CD138, CD38, BCMA (TNFRSF17), TACI, CD98, SLAM7 (CD319), FCRL5, CD63, CD74, CD59 or CD27 or any combination thereof.

In embodiments, an anchor region, which may be an antibody or part thereof, specifically binds one or more of CD138, CD38, BCMA, TACI, CD98, SLAM7 (CD319), CD27 or any combination thereof.

In embodiments, an anchor region, which may be an antibody or part thereof, specifically binds one or more of CD138, CD38, BCMA (TNFRSF17), TACI, CD98, SLAM7 (CD319), FCRL5, CD63, CD74, CD59 or CD27 or any combination thereof.

The plasma cell antigens described as targets of the anchor region of the bi-specific agent are non-limiting. A skilled person is aware of suitable other antigens that may represent plasma cell targets, and is capable of determining a plasma cell marker, or plasma cell antigen, using routine methods. In preferred embodiments the plasma cell target antigen is expressed in plasma cells to a greater extent than in other cells, for example than in other immune cells. In other preferred embodiments, the target antigen of the plasma cell is expressed in autoantibody producing plasma cells to a greater extent than in plasma cell not producing autoantibodies.

The present invention is not intended to be limited to particular plasma cell epitopes/antigens or anchor regions, e.g., binding reagent such as antibodies or fragments thereof, as long as the respective anchor region or antigen/epitope facilitates the herein intended application of the present conjugates, such as a targeting of plasma cells.

In general, antibodies directed against epitopes of plasma cells, such as CD38 or CD138, are established reagents, which are commercially available, e.g., from ThermoFisher Scientific or Miltenyi Biotec., and the skilled person is aware which antibodies or fragments thereof are suitable for targeting plasma cells. Hence, in embodiments, any commercially available anchor region, e.g., binding reagent such as antibody or fragment thereof, directed against one or more epitopes of plasma cells may be envisaged for use in the context of the present invention, as long as it facilitates the herein intended use or application of the conjugates, such as a successful targeting of plasma cells.

Conjugates comprising plasma cell-specific anchor regions have been described before, e.g., in EP 2892926 B1 or WO 2022/195238 A1. Those documents describe a conjugate suitable for the treatment of autoimmune diseases, such as rheumatic arthritis, wherein the conjugate comprises an anchor region (antibody or antigen binding fragment thereof) specific for an epitope of a plasma cell, e.g., CD138, CD38 or BCMA, and a binding region associated therewith, comprising an auto-antigen capable of binding to auto-antibodies secreted by plasma cells. However, to the knowledge of the inventors, conjugates targeting a plasma cell and comprising an interferon antigen/epitope for depletion of anti-interferon auto-antibodies, e.g., produced by plasma cells, have not been described or suggested before.

In embodiments, an anchor region is a polypeptide which consists of, or comprises, an antibody or antigen binding fragment thereof. In one aspect the antibody or binding fragment thereof is capable of binding to an antigen located on the surface of a plasma cell.

In embodiments, an anchor region may comprise an antibody or an antigen binding fragment thereof, for example may comprise a bivalent fragment such as (Fab′) 2, monovalent fragments such as Fab, single chain antibodies, Fv fragments including single chain Fv (scFv), single domain antibodies, multivalent single chain antibodies, nanobodies, diabodies, triabodies, and the like that bind specifically with an antigen. Antibody binding fragments can include polyclonal and monoclonal antibodies or antigen binding fragments thereof. Antibodies or fragments may be of any class such as IgG, IgM, IgA, IgE or IgD. The antibody or fragment thereof may be a bispecific antibody with specificity for two different plasma cell antigens, such as CD138 and CD38, for example. In embodiments, antibodies or antigen binding fragments may be humanized in whole or in part.

Preferably the bi-specific agent of the invention is not immunogenic in humans.

In embodiments, any mutations and/or recombinant variations of the Fc domains are preferably also not immunogenic in humans (e.g., such as LALAPA, ThioMab).

The person skilled in the art is familiar with methods for generation of antibodies and fragments thereof which are specific for a given target. Antibodies are readily created in animals such as rabbits or mice by immunization with the gene product, or a fragment thereof. Immunized mice can provide sources of B cells for the manufacture of hybridomas, which in turn are cultured to produce large quantities of monoclonal antibodies. These antibodies, and the nucleic acids encoding them, can be used to generate the bi-specific agent according to the present invention. For example, in embodiments a nucleic acid encoding an antibody or part thereof, which forms the anchor region, can be cloned and then ligated to a nucleic acid encoding a binding region, followed by expression in an appropriate expression system to generate the bi-specific agent of the invention.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanised antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:77 0-7 '83 (1992)).

Likewise, techniques for humanizing antibodies are also well known in the art, as are techniques for the production of fully humanized antibodies in mice.

In one aspect the anchor region comprises a variable lymphocyte receptor (VLR) or antigen binding part thereof, for example a VLR specific for a plasma cell antigen, (see Annu Rev Immunol. 2012; 30:203-20. Epub 2012 Jan. 3. VLR-based adaptive immunity.Boehm T, McCurley N, Sutoh Y, Schorpp M, Kasahara M, Cooper MD.)

In embodiments the features disclosed herein in respect of the anchor region apply equally to the bi-specific agent comprising the anchor region in association with a binding region.

In embodiments of the bi-specific agent according to the invention the anchor region is conjugated to the binding region by a covalent or non-covalent conjugation, bond and/or linker, or the anchor region and binding region are part of the same polypeptide (fusion protein).

In embodiments the anchor region of a bi-specific agent according to the invention may be associated to the binding region by chemical cross linking. In other embodiments the two regions may be associated by protein-protein interactions, such as leucine zippers. In other embodiments, the bi-specific agent may be a fusion protein comprising an anchor region and a binding region which form a part of the same polypeptide chain. In embodiments, the binding region is associated with the anchor region by a covalent bond. In embodiments, the binding region is associated with the anchor region directly, or indirectly, for example, by a linker. A linker may be included in embodiments where it is desired to introduce a spatial separation between the anchor and binding regions, or to introduce some other functionality. In embodiments, the linker may be, for example, a protein linker, a synthetic linker, a polymeric linker or any other suitable linker. In embodiments, the linker may be an amino acid sequence of, for example, 5-500 amino acids (aa). In embodiments, the anchor region and binding region each comprise a part of a linker, for example, the two regions may be linked via a biotin-streptavidin linkage, in which each one of the binding and anchor regions comprise a component of that pair, and the components associate in vivo or in vitro. In embodiments the association of the binding region with the anchor region is detectable at the surface of the plasma cell in vivo.

In some embodiments the binding region and the anchor region may be provided or administered together in associated form, for example, as a single polypeptide, in other embodiments the binding region and anchor region are provided or administered separately such that they form an association or complex at the surface of the plasma cell. For example, in embodiments the anchor could be an antibody, and the binding region itself could comprise an antibody portion specific for that first (anchor) antibody. In another embodiments the bi-specific agent may be provided as a nucleic acid such as DNA or RNA, which may be provided or administrated and subsequently expressed to form a protein constituting a bi-specific agent according the invention.

In another aspect the invention thus also relates to nucleic acids and/or vectors such as expression vectors comprising respective nucleic acid sequences and further to cells containing said nucleic acids and/or vectors.

In another aspect the present invention relates to a nucleic acid molecule encoding the bi-specific agent according to the invention.

In embodiments, the binding region is capable of binding to a specific autoantibody, such as a plasma cell autoantibody. Preferably to an (auto-) antibody released by the cell to which the anchor region is capable of binding. In embodiments, when used in the treatment of a medical condition the binding region of a bi-specific agent will not interact with all antibodies in the body of the patient receiving the treatment. In embodiments a binding region is not an antibody or part thereof directed to the constant region or other shared regions of another antibody or part thereof. In embodiments a binding region interacts with the antibody product of one plasma cell or cell line preferred over interactions with antibodies from other plasma cells.

In one aspect the binding region displays preferential binding to an antibody which is an antibody against an autoantigen, an antigen related to therapy resistance, or an allergen.

In a similar way to the interaction between anchor and plasma cell, the interaction between the binding region and plasma cell antibody may have an affinity constant in the range of 10⁶ to 10⁹, or may be greater than 10⁶, greater than 10⁷, greater than 10⁸ or greater than 10⁹.

In embodiments reference herein to the ‘binding region mediating specific binding to a plasma antibody’ can mean that the binding region can serve, for example, as a ligand, antigen or epitope (which may also be referred as a target) for a (auto-) antibody released from a plasma cell. Thus, in one embodiment, a binding region is a target for a plasma antibody. In embodiments a binding region may be bound by the variable region of a secreted antibody, such as the CDRs of the antibody released by the plasma cell.

In embodiments, a binding region can bind to a specific plasma cell antibody ligand. In embodiments a binding region may comprise an antibody, or antigen binding fragment thereof, which is capable of binding to a specific antibody released by a plasma cell, in one aspect being specific for the variable portion, or part thereof, of that plasma antibody.

In embodiments of the bi-specific agent according to the invention the binding region comprises or consists of an amino acid sequence according to SEQ ID NO 1-75, or autoantigenic fragment(s) thereof.

In embodiments, the binding region comprises or consists of an epitope of an interferon, an interferon receptor or combination thereof, or autoantigenic fragment(s) thereof.

In embodiments, an interferon (IFN) is selected from type 1 (I) interferons, type 2 (II) interferons and type 3 (III) interferons.

In embodiments of the bi-specific agent according to the invention the binding region comprises or consists of an amino acid sequence of a type 1 (I) interferon, a type 2 (II) interferon or a type 3 (III) interferon, or autoantigenic fragment(s) thereof.

In embodiments type 1 interferons are selected from interferon α (IFN alpha), interferon β (IFN beta), interferon ε (IFN epsilon), interferon κ (IFN kappa) and interferon ω (IFN omega).

In embodiments type 1 (I) interferons are selected from interferon alpha 1, interferon alpha 2, interferon alpha 4, interferon alpha 5, interferon alpha 6, interferon alpha 7, interferon alpha 8, interferon alpha 10, interferon alpha 13, interferon alpha 14, interferon alpha 16, interferon alpha 17, interferon alpha 21, interferon beta 1, interferon beta 3, interferon epsilon, interferon kappa and interferon omega 1.

In embodiments type 1 (I) interferons are selected from interferon alpha 1, interferon alpha 2, interferon alpha 4, interferon alpha 5, interferon alpha 6-1, interferon alpha 6-2, interferon alpha 7, interferon alpha 8, interferon alpha 10, interferon alpha 13-1, interferon alpha 13-2, interferon alpha 14, interferon alpha 16, interferon alpha 17, interferon alpha 21, interferon beta 1, interferon beta 3, interferon epsilon, interferon kappa and interferon omega 1.

In embodiments type 1 (I) interferons are selected from interferon alpha 1, interferon alpha 2, interferon alpha 4, interferon alpha 5, interferon alpha 6-1, interferon alpha 6-2, interferon alpha 7, interferon alpha 8, interferon alpha 10, interferon alpha 13-1, interferon alpha 13-2, interferon alpha 14, interferon alpha 16, interferon alpha 17, interferon alpha 21, interferon beta 1, interferon beta 2 (IL6-1), interferon beta 2 (IL6-2), interferon beta 2 (IL6-3), interferon beta 2 (IL6-4), interferon beta 2 (IL6-5), interferon beta 2 (IL6-6), interferon beta 2 (IL6-7), interferon beta 2 (IL6-9), interferon epsilon, interferon kappa and interferon omega 1.

In embodiments a type 2 (II) interferon is an interferon γ (IFN gamma).

In embodiments a type 3 (III) interferon is an interferon λ (IFN lambda).

In embodiments type 3 (III) interferons are selected from interferon lambda 1, interferon lambda 2, interferon lambda 3-1, interferon lambda 3-2, interferon lambda 4-1, interferon lambda 4-2 and interferon lambda 4-3.

In embodiments the binding region of the bi-specific agent comprises or consists of an amino acid sequence of interferon alpha 1, interferon alpha 2, interferon alpha 4, interferon alpha 5, interferon alpha 6-1, interferon alpha 6-2, interferon alpha 7, interferon alpha 8, interferon alpha 10, interferon alpha 13-1, interferon alpha 13-2, interferon alpha 14, interferon alpha 16, interferon alpha 17, interferon alpha 21, interferon beta 1, interferon beta 2 (IL6-1), interferon beta 2 (IL6-2), interferon beta 2 (IL6-3), interferon beta 2 (IL6-4), interferon beta 2 (IL6-5), interferon beta 2 (IL6-6), interferon beta 2 (IL6-7), interferon beta 2 (IL6-9), interferon beta 3, interferon epsilon, interferon kappa and interferon omega 1, IFN-γ (IFN gamma), interferon lambda 1, interferon lambda 2, interferon lambda 3-1, interferon lambda 3-2, interferon lambda 4-1, interferon lambda 4-2 or interferon lambda 4-3, or autoantigenic fragment(s) thereof.

In embodiments an interferon receptor is selected from interferon type 1 (1) receptor, an interferon type 2 (II) receptor and an interferon type 3 (III) receptor.

In embodiments the binding region of the bi-specific agent comprises or consists of an amino acid sequence of an interferon type 1 (I) receptor, an interferon type 2 (II) receptor or an interferon type 3 (III) receptor, or autoantigenic fragment(s) thereof.

In embodiments the interferon receptor (IFNR) is selected from interferon alpha/beta receptor, interferon gamma receptor, and interferon lambda receptor. In preferred embodiments the interferon alpha/beta receptor (IFNAR) binds to all type 1 (I) interferons.

In embodiments the interferon receptor is selected from Interferon alpha receptor 1, interferon alpha receptor 2, interferon gamma receptor 1, interferon gamma receptor 2, and interferon lambda receptor 1.

In embodiments the interferon receptor is selected from interferon alpha receptor 1 transcript variant 1, interferon alpha receptor 1 transcript variant 2, interferon alpha receptor 1 transcript variant 3, interferon alpha receptor 1 transcript variant 4, interferon alpha receptor 1 transcript variant 5, interferon alpha receptor 1 transcript variant 6, interferon alpha receptor 1 transcript variant 7, interferon alpha receptor 1 transcript variant 8, interferon alpha receptor 2 transcript variant 1, interferon alpha receptor 2 transcript variant 2, interferon alpha receptor 2 transcript variant 3, interferon alpha receptor 2 transcript variant 4, interferon alpha receptor 2 transcript variant 5, interferon alpha receptor 2 transcript variant 6, interferon alpha receptor 2 transcript variant 7, interferon alpha receptor 2 transcript variant 8, interferon gamma receptor 1 transcript variant 1, interferon gamma receptor 1 transcript variant 2, interferon gamma receptor 1 transcript variant 3, interferon gamma receptor 1 transcript variant 4, interferon gamma receptor 1 transcript variant 5, interferon gamma receptor 1 transcript variant 6, interferon gamma receptor 1 transcript variant 7, interferon gamma receptor 1 transcript variant 8, interferon gamma receptor 1 transcript variant 9, interferon gamma receptor 1 transcript variant 10, interferon gamma receptor 1 transcript variant 11, interferon gamma receptor 1 transcript variant 12, interferon gamma receptor 1 transcript variant 13, interferon gamma receptor 1 transcript variant 14, interferon gamma receptor 1 transcript variant 15, interferon gamma receptor 2 transcript variant 1, interferon gamma receptor 2 transcript variant 2, interferon gamma receptor 2 transcript variant 3, interferon gamma receptor 2 transcript variant 4, interferon lambda receptor 1 transcript variant 1, interferon lambda receptor 1 transcript variant 2, interferon lambda receptor 1 transcript variant 3, interferon lambda receptor 1 transcript variant 4 and interferon lambda receptor 1 transcript variant 5.

In embodiments, the binding region comprises or consists of an amino acid sequence comprising preferably at least a binding epitope of autoantibodies, also known as an autoepitope, e.g., any part of an autoantigen directly bound by an autoantibody, such as the amino acid sequences marked by underlining in the following Table 1. In embodiments, the autoantigenic fragments of the amino acid sequences SEQ ID 1-75 in Table 1 comprise preferably at least one binding epitope of autoantibodies, such as the amino acid sequences marked by underlining in the following Table 1.

In embodiments, the amino acid sequence of the binding epitopes of autoantibodies comprised within the binding region does not comprise sequence variation to the amino acid sequences given in Table 1. In embodiments, the amino acid sequence (comprised within the binding region) outside of the binding epitopes of autoantibodies may comprise certain sequence variations over the amino acid sequences given in Table 1. The autoantigenic component of the bispecific binding reagent may therefore, in embodiments, comprise sequence variants of the specific sequences presented in Table 1, whereby the underlined autoepitope sequences show no sequence variation from the specific sequence recited below, whereby the additional (flanking) amino acid sequence of the autoantigen outside the autoepitope may show sequence variation from the specific listed sequence, for example the varied sequence outside the specific autoepitope may exhibit at least 70%, preferably 80%, 85%, 90% or more preferably 95% sequence identity to the specific recited sequence from Table 1.

The following table 1 comprises a non-limiting and exemplary list of amino acid sequences that may be comprised by or represent the binding region of the bi-specific agent according to the invention. In embodiments the binding region of the bi-specific agent may comprise or represent an epitope which might only be a short amino acid sequence or epitope comprised by the following sequences or be a fraction of the following sequences.

TABLE 1
The table lists non-limiting examples for amino acid sequences that may be comprised
by or represent the binding region of the bi-specific agent according to the invention.
In embodiments the epitopes comprised by or representing a binding region of the bi-
specific agent might also only be a part or a shorter fragment of the following
sequences. Epitopes recognized by existing/known monoclonal antibodies are underlined
in the following and functionally important epitopes or epitopes of interest are
underlined and italic/bold in the following sequences. Such underlined sequences (and
fragments or variants thereof) may in embodiments be the epitope of the binding region.

| | | SEQ ID |
|---|---|---|
| | | NO. |
| Type I Interferons | | |
| interferon | MASPFALLMVLVVLSCKSSCSLGCDLPETHSLDNRRTLMLLAQMSRI | 1 |
| alpha 1 | SPSSCLMDRHDFGFPQEEFDGNQFQKAPAISVLHELIQQIFNLFTTK | |
| | DSSAAWDEDLLDKFCTELYQQLNDLEACVMQEERVGETPLMNADSIL | |
| | AVKKYFRRITLYLTEKKYSPCAWEVVRAEIMRSLSLSTNLQERLRRK | |
| | E | |
| interferon | MALTFALLVALLVLSCKSSCSVGCDLPQTHSLGSRRTLMLLAQMRRI | 2 |
| alpha 2 | SLFSCLKDRHDFGFPQEEFGNQFQKAETIPVLHEMIQQIFNLFSTKD | |
| | SSAAWDETLLDKFYTELYQQLNDLEACVIQGVGVTETPLMKEDSILA | |
| | VRKYFQRITLYLKEKKYSPCAWEVVRAEIMRSFSLSTNLQESLRSKE | |
| interferon | MALSFSLLMAVLVLSYKSICSLGCDLPQTHSLGNRRALILLAQMGRI | 3 |
| alpha 4 | SHFSCLKDRHDFGFPEEEFDGHQFQKAQAISVLHEMIQQTFNLFSTE | |
| | DSSAAWEQSLLEKFSTELYQQLNDLEACVIQEVGVEETPLMNEDSIL | |
| | AVRKYFQRITLYLTEKKYSPCAWEVVRAEIMRSLSFSTNLQKRLRRK | |
| | D | |
| interferon | MALPFVLLMALVVLNCKSICSLGCDLPQTHSLSNRRTLMIMAQMGRI | 4 |
| alpha 5 | SPFSCLKDRHDFGFPQEEFDGNQFQKAQAISVLHEMIQQTFNLFSTK | |
| | DSSATWDETLLDKFYTELYQQLNDLEACMMQEVGVEDTPLMNVDSIL | |
| | TVRKYFQRITLYLTEKKYSPCAWEVVRAEIMRSFSLSANLQERLRRK | |
| | E | |
| interferon | TMALPFALLMALVVLSCKSSCSLDCDLPQTHSLGHRRTMMLLAQMRR | 5 |
| alpha 6-1 | ISLFSCLKDRHDFRFPQEEFDGNQFQKAEAISVLHEVIQQTFNLFST | |
| | KDSSVAWDERLLDKLYTELYQQLNDLEACVMQEVWVGGTPLMNEDSI | |
| | LAVRKYFQRITLYLTEKKYSPCAWEVVRAEIMRSFSSSRNLQERLRR | |
| | KE | |
| interferon | MALPFALLMALVVLSCKSSCSLDCDLPQTHSLGHRRTMMLLAQMRRI | 6 |
| alpha 6-2 | SLFSCLKDRHDFRFPQEEFDGNQFQKAEAISVLHEVIQQTFNLFSTK | |
| | DSSVAWDERLLDKLYTELYQQLNDLEACVMQEVWVGGTPLMNEDSIL | |
| | AVRKYFQRITLYLTEKKYSPCAWEVVRAEIMRSFSSSRNLQERLRRK | |
| | E | |
| interferon | MARSFSLLMVVLVLSYKSICSLGCDLPQTHSLRNRRALILLAQMGRI | 7 |
| alpha 7 | SPFSCLKDRHEFRFPEEEFDGHQFQKTQAISVLHEMIQQTFNLFSTE | |
| | DSSAAWEQSLLEKFSTELYQQLNDLEACVIQEVGVEETPLMNEDFIL | |
| | AVRKYFQRITLYLMEKKYSPCAWEVVRAEIMRSFSFSTNLKKGLRRK | |
| | D | |
| interferon | MALTFYLLVALVVLSYKSFSSLGCDLPQTHSLGNRRALILLAQMRRI | 8 |
| alpha 8 | SPFSCLKDRHDFEFPQEEFDDKQFQKAQAISVLHEMIQQTFNLFSTK | |
| | DSSAALDETLLDEFYIELDQQLNDLESCVMQEVGVIESPLMYEDSIL | |
| | AVRKYFQRITLYLTEKKYSSCAWEVVRAEIMRSFSLSINLQKRLKSK | |
| | E | |
| interferon | MALSFSLLMAVLVLSYKSICSLGCDLPQTHSLGNRRALILLGQMGRI | 9 |
| alpha 10 | SPFSCLKDRHDFRIPQEEFDGNQFQKAQAISVLHEMIQQTFNLFSTE | |
| | DSSAAWEQSLLEKFSTELYQQLNDLEACVIQEVGVEETPLMNEDSIL | |
| | AVRKYFQRITLYLIERKYSPCAWEVVRAEIMRSLSFSTNLQKRLRRK | |
| | D | |
| interferon | MASPFALLMALVVLSCKSSCSLGCDLPETHSLDNRRTLMLLAQMSRI | 10 |
| alpha 13-1 | SPSSCLMDRHDFGFPQEEFDGNQFQKAPAISVLHELIQQIFNLFTTK | |
| | DSSAAWDEDLLDKFCTELYQQLNDLEACVMQEERVGETPLMNADSIL | |
| | AVKKYFRRITLYLTEKKYSPCAWEVVRAEIMRSLSLSTNLQERLRRK | |
| | F | |
| interferon | MMASPFALLMALVVLSCKSSCSLGCDLPETHSLDNRRTLMLLAQMSR | 11 |
| alpha 13-2 | ISPSSCLMDRHDFGFPQEEFDGNQFQKAPAISVLHELIQQIFNLETT | |
| | KDSSAAWDEDLLDKFCTELYQQLNDLEACVMQEERVGETPLMNADSI | |
| | LAVKKYFRRITLYLTEKKYSPCAWEVVRAEIMRSLSLSTNLQERLRR | |
| | KE | |
| interferon | MALPFALMMALVVLSCKSSCSLGCNLSQTHSLNNRRTLMLMAQMRRI | 12 |
| alpha 14 | SPFSCLKDRHDFEFPQEEFDGNQFQKAQAISVLHEMMQQTFNLFSTK | |
| | NSSAAWDETLLEKFYIELFQQMNDLEACVIQEVGVEETPLMNEDSIL | |
| | AVKKYFQRITLYLMEKKYSPCAWEVVRAEIMRSLSFSTNLQKRLRRK | |
| | D | |
| interferon | MALSFSLLMAVLVLSYKSICSLGCDLPQTHSLGNRRALILLAQMGRI | 13 |
| alpha 16 | SHFSCLKDRYDFGFPQEVEDGNQFQKAQAISAFHEMIQQTFNLFSTK | |
| | DSSAAWDETLLDKFYIELFQQLNDLEACVTQEVGVEEIALMNEDSIL | |
| | AVRKYFQRITLYLMGKKYSPCAWEVVRAEIMRSFSFSTNLQKGLRRK | |
| | D | |
| interferon | MALSFSLLMAVLVLSYKSICSLGCDLPQTHSLGNRRALILLAQMGRI | 14 |
| alpha 17 | SPFSCLKDRHDFGLPQEEFDGNQFQKTQAISVLHEMIQQTFNLFSTE | |
| | DSSAAWEQSLLEKFSTELYQQLNNLEACVIQEVGMEETPLMNEDSIL | |
| | AVRKYFQRITLYLTEKKYSPCAWEVVRAEIMRSLSFSTNLQKILRRK | |
| | D | |
| interferon | MALSFSLLMAVLVLSYKSICSLGCDLPQTHSLGNRRALILLAQMGRI | 15 |
| alpha 21 | SPFSCLKDRHDFGFPQEEFDGNQFQKAQAISVLHEMIQQTFNLFSTK | |
| | DSSATWEQSLLEKFSTELNQQLNDLEACVIQEVGVEETPLMNVDSIL | |
| | AVKKYFQRITLYLTEKKYSPCAWEVVRAEIMRSFSLSKIFQERLRRK | |
| | E | |
| interferon | MTNKCLLQIALLLCFSTTALSMSYNLLGFLQRSSNFQCQKLLWQLNG | 16 |
| beta 1 | RLEYCLKDRMNFDIPEEIKQLQQFQKEDAALTIYEMLQNIFAIFRQD | |
| | SSSTGWNETIVENLLANVYHQINHLKTVLEEKLEKEDFTRGKLMSSL | |
| | HLKRYYGRILHYLKAKEYSHCAWTIVRVEILRNFYFINRLTGYLRN | |
| interferon | MNSFSTSAFGPVAFSLGLLLVLPAAFPAPVPPGEDSKDVAAPHRQPL | 17 |
| beta 2 | TSSERIDKQIRYILDGISALRKETCNKSNMCESSKEALAENNLNLPK | |
| (IL6-1) | MAEKDGCFQSGFNEETCLVKIITGLLEFEVYLEYLQNRFESSEEQAR | |
| | AVQMSTKVLIQFLQKKAKNLDAITTPDPTTNASLLTKLQAQNQWLQD | |
| | MTTHLILRSFKEFLQSSLRALRQM | |
| interferon | MNSFSTIPPGEDSKDVAAPHRQPLTSSERIDKQIRYILDGISALRKE | 18 |
| beta 2 | TCNKSNMCESSKEALAENNLNLPKMAEKDGCFQSGFNEETCLVKIIT | |
| (IL6-2) | GLLEFEVYLEYLQNRFESSEEQARAVQMSTKVLIQFLQKKAKNLDAI | |
| | TTPDPTTNASLLTKLQAQNQWLQDMTTHLILRSFKEFLQSSLRALRQ | |
| | M | |
| interferon | MCESSKEALAENNLNLPKMAEKDGCFQSGFNEETCLVKIITGLLEFE | 19 |
| beta 2 | VYLEYLQNRFESSEEQARAVQMSTKVLIQFLQKKVGVSSFPQLGVGE | |
| (IL6-3) | DRLKDSVLDNSGMQCHFQKRRLHVNKRV | |
| interferon | MNSFSTSAFGPVAFSLGLLLVLPAAFPAPVPPGEDSKDVAAPHRQPL | 20 |
| beta 2 | TSSERIDKQIRYILDGISALRKETCNKSNMCESSKEALAENNLNLPK | |
| (IL6-4) | MAEKDGCFQSGFNEETCLVKIITGLLEFEVYLEYLQNRFESSEEQAR | |
| | AVQMSTKVLIQFLQKKAKNLDAITTPDPTTNASLLTKLQAQNQWLQD | |
| | MTTHLILRSFKEFLQSSLRALRQM | |
| interferon | MNSFSTSAFGPVAFSLGLLLVLPAAFPAPVPPGEDSKDVAAPHRQPL | 21 |
| beta 2 | TSSERIDKQIRYILDGISALRKETCNKSNMCESSKEALAENNLNLPK | |
| (IL6-5) | MAEKDGCFQSGFNEETCLVKIITGLLEFEVYLEYLQNRFESSEEQAR | |
| | AVQMSTKVLIQFLQKKVGVSSFPQLGVGEDRLKDSVLDNSGMQCHFQ | |
| | KRRLHVNKRV | |
| interferon | MCESSKEALAENNLNLPKMAEKDGCFQSGFNEETCLVKIITGLLEFE | 22 |
| beta 2 | VYLEYLQNRFESSEEQARAVQMSTKVLIQFLQKKAKNLDAITTPDPT | |
| (IL6-6) | TNASLLTKLQAQNQWLQDMTTHLILRSFKEFLQSSLRALRQM | |
| interferon | MNSFSTSAFGPVAFSLGLLLVLPAAFPAPVPPGEDSKDVAAPHRQPL | 23 |
| beta 2 | TSSERIDKQIRYILDGISALRKETCNKSNMCESSKEALAENNLNLPK | |
| (IL6-7) | MAEKDGCFQSGFNEETCLVKIITGLLEFEVYLEYLQNRFESSEEQAR | |
| | AVQMSTKVLIQ | |
| interferon | MNSFSTSKCRKSLALELPAAVEPCVREGCVAQGGLAGGQQQRQAPSC | 24 |
| beta 2 | AVSSPLRSLPSGTGAFGPVAFSLGLLLVLPAAFPAPVPPGEDSKDVA | |
| (IL6-9) | APHRQPLTSSERIDKQIRYILDGISALRKETCNKSNMCESSKEALAE | |
| | NNLNLPKMAEKDGCFQSGFNEETCLVKIITGLLEFEVYLEYLQNRFE | |
| | SSEEQARAVQMSTKVLIQFLQKKAKNLDAITTPDPTTNASLLTKLQA | |
| | QNQWLQDMTTHLILRSFKEFLQSSLRALRQM | |
| interferon | MIIKHFFGTVLVLLASTTIFSLDLKLIIFQQRQVNQESLKLLNKLQT | 25 |
| epsilon | LSIQQCLPHRKNFLLPQKSLSPQQYQKGHTLAILHEMLQQIFSLFRA | |
| | NISLDGWEENHTEKFLIQLHQQLEYLEALMGLEAEKLSGTLGSDNLR | |
| | LQVKMYFRRIHDYLENQDYSTCAWAIVQVEISRCLFFVFSLTEKLSK | |
| | QGRPLNDMKQELTTEFRSPR | |
| interferon | MSTKPDMIQKCLWLEILMGIFIAGTLSLDCNLLNVHLRRVTWQNLRH | 26 |
| kappa | LSSMSNSFPVECLRENIAFELPQEFLQYTQPMKRDIKKAFYEMSLQA | |
| | FNIFSQHTFKYWKERHLKQIQIGLDQQAEYLNQCLEEDKNENEDMKE | |
| | MKENEMKPSEARVPQLSSLELRRYFHRIDNFLKEKKYSDCAWEIVRV | |
| | EIRRCLYYFYKFTALFRRK | |
| interferon | MALLFPLLAALVMTSYSPVGSLGCDLPQNHGLLSRNTLVLLHQMRRI | 27 |
| omega 1 | SPFLCLKDRRDFRFPQEMVKGSQLQKAHVMSVLHEMLQQIFSLFHTE | |
| | RSSAAWNMTLLDQLHTGLHQQLQHLETCLLQVVGEGESAGAISSPAL | |
| | TLRRYFQGIRVYLKEKKYSDCAWEVVRMEIMKSLFLSTNMQERLRSK | |
| | DRDLGSS | |

Type II Interferon

| | | |
|---|---|---|
| interferon | MKYTSYILAFQLCIVLGSLGCYCQDPYVKEAENLKKYFNAGHSDVAD | 28 |
| gamma | NGTLFLGILKNWKEESDRKIMQSQIVSFYFKLFKNFKDDQSIQKSVE | |
| | TIKEDMNVKFFNSNKKKRDDFEKLTNYSVTDLNVQRKAIHELIQVMA | |
| | ELSPAAKTGKRKRSQMLFRGRRASQ | |

Type III Interferons

| | | |
|---|---|---|
| interferon | MAAAWTVVLVTLVLGLAVAGPVPTSKPTTTGKGCHIGRFKSLSPQEL | 29 |
| lambda 1 | ASFKKARDALEESLKLKNWSCSSPVFPGNWDLRLLQVRERPVALEAE | |
| | LALTLKVLEAAAGPALEDVLDQPLHTLHHILSQLQACIQPQPTAGPR | |
| | PRGRLHHWLHRLQEAPKKESAGCLEASVTFNLFRLLTRDLKYVADGN | |
| | LCLRTSTHPEST | |
| interferon | MKLDMTGDCTPVLVLMAAVLTVTGAVPVARLHGALPDARGCHIAQFK | 30 |
| lambda 2 | SLSPQELQAFKRAKDALEESLLLKDCRCHSRLFPRTWDLRQLQVRER | |
| | PMALEAELALTLKVLEATADTDPALVDVLDQPLHTLHHILSQFRACI | |
| | QPQPTAGPRTRGRLHHWLYRLQEAPKKESPGCLEASVTFNLFRLLTR | |
| | DLNCVASGDLCV | |
| interferon | MTGDCMPVLVLMAAVLTVTGAVPVARLRGALPDARGCHIAQFKSLSP | 31 |
| lambda 3-1 | QELQAFKRAKDALEESLLLKDCKCRSRLFPRTWDLRQLQVRERPVAL | |
| | EAELALTLKVLEATADTDPALGDVLDQPLHTLHHILSQLRACIQPQP | |
| | TAGPRTRGRLHHWLHRLQEAPKKESPGCLEASVTFNLFRLLTRDLNC | |
| | VASGDLC | |
| interferon | MKLDMTGDCMPVLVLMAAVLTVTGAVPVARLRGALPDARGCHIAQFK | 32 |
| lambda 3-2 | SLSPQELQAFKRAKDALEESLLLKDCKCRSRLFPRTWDLRQLQVRER | |
| | PVALEAELALTLKVLEATADTDPALGDVLDQPLHTLHHILSQLRACI | |
| | QPQPTAGPRTRGRLHHWLHRLQEAPKKESPGCLEASVTFNLFRLLTR | |
| | DLNCVASGDLCV | |
| interferon | MRPSVWAAVAAGLWVLCTVIAEGPPALPALALPLAGAPDAGGCQGAE | 33 |
| lambda 4-1 | GPLRGRGAELGAAQLLLPPQEGSSAAIVLRSAPPRGPGHRGRPGSAQ | |
| | RPAPLGAAPRRRPDPGAAGGRGEGCGGLPAGTARLLQEGPRGPEEAS | |
| | QTPESGLASVPQSQRGLQPPAPAHVGAPAGCTLWALPL | |
| interferon | MRPSVWAAVAAGLWVLCTVIAEGPPALPALALPLAGAPDAGGCQGAE | 34 |
| lambda 4-2 | GPLPAGTARLLQEGPRGPEEASQTPESGLASVPQSQRGLQPPAPAHV | |
| | GAPAGCTLWALPL | |
| interferon | MRPSVWAAVAAGLWVLCTVIAEGPPALPALALPLAGAPDAGGCQGAE | 35 |
| lambda 4-3 | GPLRGRGAELGAAQLLLPPQEGSSAAIAAGTARLLQEGPRGPEEASQ | |
| | TPESGLASVPQSQRGLQPPAPAHVGAPAGCTLWALPL | |

Interferons receptors

| | | |
|---|---|---|
| Interferon | MMVVLLGATTLVLVAVAPWVLSAAAGGKNLKSPQKVEVDIIDDNFIL | 36 |
| alpha | RWNRSDESVGNVTFSFDYQKTGMDNWIKLSGCQNITSTKCNFSSLKL | |
| receptor 1 | NVYEEIKLRIRAEKENTSSWYEVDSFTPFRKAQIGPPEVHLEAEDKA | |
| transcript | IVIHISPGTKDSVMWALDGLSFTYSLVIWKNSSGVEERIENIYSRHK | |
| variant1 | IYKLSPETTYCLKVKAALLTSWKIGVYSPVHCIKTTVENELPPPENI | |
| | EVSVQNQNYVLKWDYTYANMTFQVQWLHAFLKRNPGNHLYKWKQIPD | |
| | CENVKTTQCVFPQNVFQKGIYLLRVQASDGNNTSFWSEEIKFDTEIQ | |
| | AFLLPPVENIRSLSDSFHIYIGAPKQSGNTPVIQDYPLIYEIIFWEN | |
| | TSNAERKIIEKKTDVTVPNLKPLTVYCVKARAHTMDEKLNKSSVFSD | |
| | AVCEKTKPGNTSKIWLIVGICIALFALPFVIYAAKVFLRCINYVFFP | |
| | SLKPSSSIDEYFSEQPLKNLLLSTSEEQIEKCFIIENISTIATVEET | |
| | NQTDEDHKKYSSQTSQDSGNYSNEDESESKTSEELQQDFV | |
| Interferon | MMVVLLGATTLVLVAVAPWVLSAAAGGKNLKSPQKVEVDIIDDNFIL | 37 |
| alpha | RWNRSDESVGNVTFSFDYQKTGMDNWIKLSGCQNITSTKCNFSSLKL | |
| receptor 1 | NVYEEIKLRIRAEKENTSSWYEVDSFTPFRKVENELPPPENIEVSVQ | |
| transcript | NQNYVLKWDYTYANMTFQVQWLHAFLKRNPGNHLYKWKQIPDCENVK | |
| variant 2 | TTQCVFPQNVFQKGIYLLRVQASDGNNTSFWSEEIKFDTEIQAFLLP | |
| | PVFNIRSLSDSFHIYIGAPKQSGNTPVIQDYPLIYEIIFWENTSNAE | |
| | RKIIEKKTDVTVPNLKPLTVYCVKARAHTMDEKLNKSSVFSDAVCEK | |
| | TKPGNTSKIWLIVGICIALFALPFVIYAAKVFLRCINYVFFPSLKPS | |
| | SSIDEYFSEQPLKNLLLSTSEEQIEKCFIIENISTIATVEETNQTDE | |
| | DHKKYSSQTSQDSGNYSNEDESESKTSEELQQDFV | |
| Interferon | MDNWIKLSGCQNITSTKCNFSSLKLNVYEEIKLRIRAEKENTSSWYE | 38 |
| alpha | VDSFTPFRKAQIGPPEVHLEAEDKAIVIHISPGTKDSVMWALDGLSF | |
| receptor 1 | TYSLVIWKNSSGVEERIENIYSRHKIYKLSPETTYCLKVKAALLTSW | |
| transcript | KIGVYSPVHCIKTTVENELPPPENIEVSVQNQNYVLKWDYTYANMTF | |
| variant 3 | QVQWLHAFLKRNPGNHLYKWKQIPDCENVKTTQCVFPQNVFQKGIYL | |
| | LRVQASDGNNTSFWSEEIKFDTEIQAFLLPPVENIRSLSDSFHIYIG | |
| | APKQSGNTPVIQDYPLIYEIIFWENTSNAERKIIEKKTDVTVPNLKP | |
| | LTVYCVKARAHTMDEKLNKSSVFSDAVCEKTKPGNTSKIWLIVGICI | |
| | ALFALPFVIYAAKVFLRCINYVFFPSLKPSSSIDEYFSEQPLKNLLL | |
| | STSEEQIEKCFIIENISTIATVEETNQTDEDHKKYSSQTSQDSGNYS | |
| | NEDESESKTSEELQQDFV | |
| Interferon | MMVVLLGATTLVLVAVAPWVLSAAAGGKNLKSPQKVEVDIIDDNFIL | 39 |
| alpha | RWNRSDESVGNVTFSFDYQKTGMDNWIKLSGCQNITSTKCNFSSLKL | |
| receptor 1 | NVYEEIKLRIRAEKENTSSWYEVDSFTPFRKAQIGPPEVHLEAEDKA | |
| transcript | IVIHISPGTKDSVMWALDGLSFTYSLVIWKNSSGVEERIENIYSRHK | |
| variant 4 | IYKLSPETTYCLKVKAALLTSWKIGVYSPVHCIKTTVENELPPPENI | |
| | EVSVQNQNYVLKWDYTYANMTFQVQWLHLYFRRPR | |
| Interferon | MMVVLLGATTLVLVAVAPWVLSAAAGGKNLKSPQKVEVDIIDDNFIL | 40 |
| alpha | RWNRSDESVGNVTFSFDYQKTGMDNWIKLSGCQNITSTKCNFSSLKL | |
| receptor 1 | NVYEEIKLRIRAEKENTSSWYEVDSFTPFRKAQIGPPEVHLEAEDKA | |
| transcript | IVIHISPGTKDSVMWALDGLSFTYSLVIWKNSSGVEERIENIYSRHK | |
| variant 5 | IYKLSPETTYCLKVKAALLTSWKIGVYSPVHCIKTTVCILGGRGRWI | |
| | T | |
| Interferon | MMVVLLGATTLVLVAVAPWVLSAAAGGKNLKSPQKVEVDIIDDNFIL | 41 |
| alpha | RWNRSDESVGNVTFSFDYQKTGMDNWIKLSGCQNITSTKCNFSSLKL | |
| receptor 1 | NVYEEIKLRIRAEKENTSSWYEVDSFTPFRKGKD | |
| transcript | | |
| variant 6 | | |
| Interferon | MDNWIKLSGCQNITSTKCNFSSLKLNVYEEIKLRIRAEKENTSSWYE | 42 |
| alpha | VDSFTPFRKAQIGPPEVHLEAEDKAIVIHISPGTKDSVMWALDGLSF | |
| receptor 1 | TYSLVIWKNSSGVEERIENIYSRHKIYKLSPETTYCLKVKAALLTSW | |
| transcript | KIGVYSPVHCIKTTVENELPPPENIEVSVQNQNYVLKWDYTYANMTF | |
| variant 7 | QVQWLHAFLKRNPGNHLYKWKQIPDCENVKTTQCVFPQNVFQKGIYL | |
| | LRVQASDGNNTSFWSEEIKFDTEIQAFLLPPVENIRSLSDSFHIYIG | |
| | APKQSGNTPVIQDYPLIYEIIFWENTSNAERKIIEKKTDVTVPNLKP | |
| | LTVYCVKARAHTMDEKLNKSSVFSDAVCEKTKPGNTSKIWLIVGICI | |
| | ALFALPFVIYAAKVFLRCINYVFFPSLKPSSSIDEYFSEQPLKNLLL | |
| | STSEEQIEKCFIIENISTIATVEETNQTDEDHKKYSSQTSQDSGNYS | |
| | NEDESESKTSEELQQDFV | |
| Interferon | MTFQVQWLHAFLKRNPGNHLYKWKQIPDCENVKTTQCVFPQNVFQKG | 43 |
| alpha | IYLLRVQASDGNNTSFWSEEIKFDTEIQAFLLPPVENIRSLSDSFHI | |
| receptor 1 | YIGAPKQSGNTPVIQDYPLIYEIIFWENTSNAERKIIEKKTDVTVPN | |
| transcript | LKPLTVYCVKARAHTMDEKLNKSSVFSDAVCEKTKPGNTSKIWLIVG | |
| variant 8 | ICIALFALPFVIYAAKVFLRCINYVFFPSLKPSSSIDEYFSEQPLKN | |
| | LLLSTSEEQIEKCFIIENISTIATVEETNQTDEDHKKYSSQTSQDSG | |
| | NYSNEDESESKTSEELQQDFV | |
| Interferon | MLLSQNAFIFRSLNLVLMVYISLVFGISYDSPDYTDESCTFKISLRN | 44 |
| alpha | FRSILSWELKNHSIVPTHYTLLYTIMSKPEDLKVVKNCANTTRSFCD | |
| receptor 2 | LTDEWRSTHEAYVTVLEGFSGNTTLFSCSHNFWLAIDMSFEPPEFEI | |
| transcript | VGFTNHINVMVKFPSIVEEELQFDLSLVIEEQSEGIVKKHKPEIKGN | |
| variant 1 | MSGNFTYIIDKLIPNTNYCVSVYLEHSDEQAVIKSPLKCTLLPPGQE | |
| | SESAESAKIGGIITVFLIALVLTSTIVTLKWIGYICLRNSLPKVLNF | |
| | HNFLAWPFPNLPPLEAMDMVEVIYINRKKKVWDYNYDDESDSDTEAA | |
| | PRTSGGGYTMHGLTVRPLGQASATSTESQLIDPESEEEPDLPEVDVE | |
| | LPTMPKDSPQQLELLSGPCERRKSPLQDPFPEEDYSSTEGSGGRITE | |
| | NVDLNSVFLRVLDDEDSDDLEAPLMLSSHLEEMVDPEDPDNVQSNHL | |
| | LASGEGTQPTFPSPSSEGLWSEDAPSDQSDTSESDVDLGDGYIMR | |
| Interferon | MLLSQNAFIFRSLNLVLMVYISLVFGISYDSPDYTDESCTFKISLRN | 45 |
| alpha | FRSILSWELKNHSIVPTHYTLLYTIMSKPEDLKVVKNCANTTRSFCD | |
| receptor 2 | LTDEWRSTHEAYVTVLEGFSGNTTLFSCSHNFWLAIDMSFEPPEFEI | |
| transcript | VGFTNHINVMVKFPSIVEEELQFDLSLVIEEQSEGIVKKHKPEIKGN | |
| variant 2 | MSGNFTYIIDKLIPNTNYCVSVYLEHSDEQAVIKSPLKCTLLPPGQE | |
| | SEFS | |
| Interferon | MLLSQNAFIFRSLNLVLMVYISLVFGISYDSPDYTDESCTFKISLRN | 46 |
| alpha | FRSILSWELKNHSIVPTHYTLLYTIMSKPEDLKVVKNCANTTRSFCD | |
| receptor 2 | LTDEWRSTHEAYVTVLEGFSGNTTLFSCSHNFWLAIDMSFEPPEFEI | |
| transcript | VGFTNHINVMVKFPSIVEEELQFDLSLVIEEQSEGIVKKHKPEIKGN | |
| variant 3 | MSGNFTYIIDKLIPNTNYCVSVYLEHSDEQAVIKSPLKCTLLPPGQE | |
| | SESAESAKIGGIITVFLIALVLTSTIVTLKWIGYICLRNSLPKVLRQ | |
| | GLAKGWNAVAIHRCSHNALQSETPELKQSSCLSFPSSWDYKRASLCP | |
| | SD | |
| Interferon | MLLSQNAFIFRSLNLVLMVYISLVFGISYDSPDYTDESCTFKISLRN | 47 |
| alpha | FRSILSWELKNHSIVPTHYTLLYTIMSKPEDLKVVKNCANTTRSFCD | |
| receptor 2 | LTDEWRSTHEAYVTVLEGFSGNTTLFSCSHNEWLAIDMSFEPPEFEI | |
| transcript | VGFTNHINVMVKFPSIVEEELQFDLSLVIEEQSEGIVKKHKPEIKGN | |
| variant 4 | MSGNFTYIIDKLIPNTNYCVSVYLEHSDEQAVIKSPLKCTLLPPGQE | |
| | SESAESAKIGGIITVFLIALVLTSTIVTLKWIGYICLRNSLPKVLRQ | |
| | GLAKGWNAVAIHRCSHNALQSETPELKQSSCLSFPSSWDYKRASLCP | |
| | SD | |
| Interferon | MSKPEDLKVVKNCANTTRSFCDLTDEWRSTHEAYVTVLEGESGNTTL | 48 |
| alpha | FSCSHNFWLAIDMSFEPPEFEIVGFTNHINVMVKFPSIVEEELQFDL | |
| receptor 2 | SLVIEEQSEGIVKKHKPEIKGNMSGNFTYIIDKLIPNTNYCVSVYLE | |
| transcript | HSDEQAVIKSPLKCTLLPPGQESEFS | |
| variant 5 | | |
| Interferon | MSKPEDLKVVKNCANTTRSFCDLTDEWRSTHEAYVTVLEGFSGNTTL | 49 |
| alpha | FSCSHNFWLAIDMSFEPPEFEIVGFTNHINVMVKFPSIVEEELQFDL | |
| receptor 2 | SLVIEEQSEGIVKKHKPEIKGNMSGNFTYIIDKLIPNTNYCVSVYLE | |
| transcript | HSDEQAVIKSPLKCTLLPPGQESESAESAKIGGIITVFLIALVLTST | |
| variant 6 | IVTLKWIGYICLRNSLPKVLNFHNFLAWPFPNLPPLEAMDM | |
| Interferon | MPRIPGICHCKICKSQEKTLKIAKMLLSQNAFIFRSLNLVLMVYISL | 50 |
| alpha | VFGISYDSPDYTDESCTFKISLRNERSILSWELKNHSIVPTHYTLLY | |
| receptor 2 | TIMSKPEDLKVVKNCANTTRSFCDLTDEWRSTHEAYVTVLEGFSGNT | |
| transcript | TLFSCSHNFWLAIDMSFEPPEFEIVGFTNHINVMVKFPSIVEEELQF | |
| variant 7 | DLSLVIEEQSEGIVKK | |
| Interferon | MLLSQNAFIFRSLNLVLMVYISLVFGISYDSPDYTDESCTFKISLRN | 51 |
| alpha | FRSILSWELKNHSIVPTHYTLLYTIMSKPEDLKVVKNCANTTRSFCD | |
| receptor 2 | LTDEWRSTHEAYVTVLEGFSGNTTLFSCSHNFWLAIDMSFEPPEFEI | |
| transcript | VGFTNHINVMVKFPSIVEEELQFDLSLVIEEQSEGIVKKHKPEIKGN | |
| variant 8 | MSGNFTYIIDKLIPNTNYCVSVYLEHSDEQAVIKSPLKCTLLPPGQE | |
| | SESAESAKIGGIITVFLIALVLTSTIVTLKWIGYICLRNSLPKVLNF | |
| | HNFLAWPFPNLPPLEAMDMVEVIYINRKKKVWDYNYDDESDSDTEAA | |
| | PRTSGGGYTMHGLTVRPLGQASATSTESQLIDPESEEEPDLPEVDVE | |
| | LPTMPKDSPQQLELLSGPCERRKSPLQDPFPEEDYSSTEGSGGRITF | |
| | NVDLNSVFLRVLDDEDSDDLEAPLMLSSHLEEMVDPEDPDNVQSNHL | |
| | LASGEGTQPTFPSPSSEGLWSEDAPSDQSDTSESDVDLGDGYIMR | |
| Interferon | MALLFLLPLVMQGVSRAEMGTADLGPSSVPTPTNVTIESYNMNPIVY | 52 |
| gamma | WEYQIMPQVPVFTVEVKNYGVKNSEWIDACINISHHYCNISDHVGDP | |
| receptor 1 | SNSLWVRVKARVGQKESAYAKSEEFAVCRDGKIGPPKLDIRKEEKQI | |
| transcript | MIDIFHPSVFVNGDEQEVDYDPETTCYIRVYNVYVRMNGSEIQYKIL | |
| variant 1 | TQKEDDCDEIQCQLAIPVSSLNSQYCVSAEGVLHVWGVTTEKSKEVC | |
| | ITIFNSSIKGSLWIPVVAALLLFLVLSLVFICFYIKKINPLKEKSII | |
| | LPKSLISVVRSATLETKPESKYVSLITSYQPFSLEKEVVCEEPLSPA | |
| | TVPGMHTEDNPGKVEHTEELSSITEVVTTEENIPDVVPGSHLTPIER | |
| | ESSSPLSSNQSEPGSIALNSYHSRNCSESDHSRNGFDTDSSCLESHS | |
| | SLSDSEFPPNNKGEIKTEGQELITVIKAPTSFGYDKPHVLVDLLVDD | |
| | SGKESLIGYRPTEDSKEFS | |
| Interferon | MLLKSPENSLLQFQFKYGVPTPTNVTIESYNMNPIVYWEYQIMPQVP | 53 |
| gamma | VFTVEVKNYGVKNSEWIDACINISHHYCNISDHVGDPSNSLWVRVKA | |
| receptor 1 | RVGQKESAYAKSEEFAVCRDGKIGPPKLDIRKEEKQIMIDIFHPSVE | |
| transcript | VNGDEQEVDYDPETTCYIRVYNVYVRMNGSEIQYKILTQKEDDCDEI | |
| variant 2 | QCQLAIPVSSLNSQYCVSAEGVLHVWGVTTEKSKEVCITIENSSIKG | |
| | SLWIPVVAALLLFLVLSLVFICFYIKKINPLKEKSIILPKSLISVVR | |
| | SATLETKPESKYVSLITSYQPFSLEKEVVCEEPLSPATVPGMHTEDN | |
| | PGKVEHTEELSSITEVVTTEENIPDVVPGSHLTPIERESSSPLSSNQ | |
| | SEPGSIALNSYHSRNCSESDHSRNGFDTDSSCLESHSSLSDSEFPPN | |
| | NKGEIKTEGQELITVIKAPTSFGYDKPHVLVDLLVDDSGKESLIGYR | |
| | PTEDSKEFS | |
| Interferon | MALLFLLPLVMQGVSRAEMGTADLGPSSVPTPTNVTIESYNMNPIVY | 54 |
| gamma | WEYQIMPQVPVFTVEVKNYGVKARVGQKESAYAKSEEFAVCRDGKIG | |
| receptor 1 | PPKLDIRKEEKQIMIDIFHPSVFVNGDEQEVDYDPETTCYIRVYNVY | |
| transcript | VRMNGSEIQYKILTQKEDDCDEIQCQLAIPVSSLNSQYCVSAEGVLH | |
| variant 3 | VWGVTTEKSKEVCITIFNSSIKGSLWIPVVAALLLFLVLSLVFICFY | |
| | IKKINPLKEKSIILPKSLISVVRSATLETKPESKYVSLITSYQPFSL | |
| | EKEVVCEEPLSPATVPGMHTEDNPGKVEHTEELSSITEVVTTEENIP | |
| | DVVPGSHLTPIERESSSPLSSNQSEPGSIALNSYHSRNCSESDHSRN | |
| | GFDTDSSCLESHSSLSDSEFPPNNKGEIKTEGQELITVIKAPTSFGY | |
| | DKPHVLVDLLVDDSGKESLIGYRPTEDSKEFS | |
| Interferon | MCGSIVQSLVPTPTNVTIESYNMNPIVYWEYQIMPQVPVFTVEVKNY | 55 |
| gamma | GVKNSEWIDACINISHHYCNISDHVGDPSNSLWVRVKARVGQKESAY | |
| receptor 1 | AKSEEFAVCRDGKIGPPKLDIRKEEKQIMIDIFHPSVFVNGDEQEVD | |
| transcript | YDPETTCYIRVYNVYVRMNGSEIQYKILTQKEDDCDEIQCQLAIPVS | |
| variant 4 | SLNSQYCVSAEGVLHVWGVTTEKSKEVCITIFNSSIKGSLWIPVVAA | |
| | LLLFLVLSLVFICFYIKKINPLKEKSIILPKSLISVVRSATLETKPE | |
| | SKYVSLITSYQPFSLEKEVVCEEPLSPATVPGMHTEDNPGKVEHTEE | |
| | LSSITEVVTTEENIPDVVPGSHLTPIERESSSPLSSNQSEPGSIALN | |
| | SYHSRNCSESDHSRNGFDTDSSCLESHSSLSDSEFPPNNKGEIKTEG | |
| | QELITVIKAPTSFGYDKPHVLVDLLVDDSGKESLIGYRPTEDSKEFS | |
| Interferon | MNPIVYWEYQIMPQVPVFTVEVKNYGVKNSEWIDACINISHHYCNIS | 56 |
| gamma | DHVGDPSNSLWVRVKARVGQKESAYAKSEEFAVCRDGKIGPPKLDIR | |
| receptor 1 | KEEKQIMIDIFHPSVFVNGDEQEVDYDPETTCYIRVYNVYVRMNGSE | |
| transcript | IQYKILTQKEDDCDEIQCQLAIPVSSLNSQYCVSAEGVLHVWGVTTE | |
| variant 5 | KSKEVCITIFNSSIKGSLWIPVVAALLLFLVLSLVFICFYIKKINPL | |
| | KEKSIILPKSLISVVRSATLETKPESKYVSLITSYQPFSLEKEVVCE | |
| | EPLSPATVPGMHTEDNPGKVEHTEELSSITEVVTTEENIPDVVPGSH | |
| | LTPIERESSSPLSSNQSEPGSIALNSYHSRNCSESDHSRNGFDTDSS | |
| | CLESHSSLSDSEFPPNNKGEIKTEGQELITVIKAPTSFGYDKPHVLV | |
| | DLLVDDSGKESLIGYRPTEDSKEFS | |
| Interferon | MCGSIVQSLVPTPTNVTIESYNMNPIVYWEYQIMPQVPVFTVEVKNY | 57 |
| gamma | GVKNSEWIDACINISHHYCNISDHVGDPSNSLWVRVKARVGQKESAY | |
| receptor 1 | AKSEEFAVCRDGKIGPPKLDIRKEEKQIMIDIFHPSVFVNGDEQEVD | |
| transcript | YDPETTCYIRVYNVYVRMNGSEIQYKILTQKEDDCDEIQCQLAIPVS | |
| variant 6 | SLNSQYCVSAEGVLHVWGVTTEKSKEVCITIFNSSIKGSLWIPVVAA | |
| | LLLFLVLSLVFICFYIKKINPLKEKSIILPKSLISVVRSATLETKPE | |
| | SKYVSLITSYQPFSLEKEVVCEEPLSPATVPGMHTEDNPGKVEHTEE | |
| | LSSITEVVTTEENIPDVVPGSHLTPIERESSSPLSSNQSEPGSIALN | |
| | SYHSRNCSESDHSRNGFDTDSSCLESHSSLSDSEFPPNNKGEIKTEG | |
| | QELITVIKAPTSFGYDKPHVLVDLLVDDSGKESLIGYRPTEDSKEFS | |
| Interferon | MNPIVYWEYQIMPQVPVFTVEVKNYGVKNSEWIDACINISHHYCNIS | 58 |
| gamma | DHVGDPSNSLWVRVKARVGQKESAYAKSEEFAVCRDGKIGPPKLDIR | |
| receptor 1 | KEEKQIMIDIFHPSVFVNGDEQEVDYDPETTCYIRVYNVYVRMNGSE | |
| transcript | IQYKILTQKEDDCDEIQCQLAIPVSSLNSQYCVSAEGVLHVWGVTTE | |
| variant 7 | KSKEVCITIFNSSIKGSLWIPVVAALLLFLVLSLVFICFYIKKINPL | |
| | KEKSIILPKSLISVVRSATLETKPESKYVSLITSYQPFSLEKEVVCE | |
| | EPLSPATVPGMHTEDNPGKVEHTEELSSITEVVTTEENIPDVVPGSH | |
| | LTPIERESSSPLSSNQSEPGSIALNSYHSRNCSESDHSRNGFDTDSS | |
| | CLESHSSLSDSEFPPNNKGEIKTEGQELITVIKAPTSFGYDKPHVLV | |
| | DLLVDDSGKESLIGYRPTEDSKEFS | |
| Interferon | MLLKSPENSLLQFQFKYGVPTPTNVTIESYNMNPIVYWEYQIMPQVP | 59 |
| gamma | VFTVEVKNYGVKNSEWIDACINISHHYCNISDHVGDPSNSLWVRVKA | |
| receptor 1 | RVGQKESAYAKSEEFAVCRDGKIGPPKLDIRKEEKQIMIDIFHPSVF | |
| transcript | VNGDEQEVDYDPETTCYIRVYNVYVRMNGSEIQYKILTQKEDDCDEI | |
| variant 8 | QCQLAIPVSSLNSQYCVSAEGVLHVWGVTTEKSKEVCITIFNSSIKG | |
| | SLWIPVVAALLLFLVLSLVFICFYIKKINPLKEKSIILPKSLISVVR | |
| | SATLETKPESKYVSLITSYQPFSLEKEVVCEEPLSPATVPGMHTEDN | |
| | PGKVEHTEELSSITEVVTTEENIPDVVPGSHLTPIERESSSPLSSNQ | |
| | SEPGSIALNSYHSRNCSESDHSRNGFDTDSSCLESHSSLSDSEFPPN | |
| | NKGEIKTEGQELITVIKAPTSFGYDKPHVLVDLLVDDSGKESLIGYR | |
| | PTEDSKEFS | |
| Interferon | MLLKSPENSLLQFQFKYGVPTPTNVTIESYNMNPIVYWEYQIMPQVP | 60 |
| gamma | VFTVEVKNYGVKNSEWIDACINISHHYCNISDHVGDPSNSLWVRVKA | |
| receptor 1 | RVGQKESAYAKSEEFAVCRDGKIGPPKLDIRKEEKQIMIDIFHPSVF | |
| transcript | VNGDEQEVDYDPETTCYIRVYNVYVRMNGSEIQYKILTQKEDDCDEI | |
| variant 9 | QCQLAIPVSSLNSQYCVSAEGVLHVWGVTTEKSKEVCITIFNSSIKG | |
| | SLWIPVVAALLLFLVLSLVFICFYIKKINPLKEKSIILPKSLISVVR | |
| | SATLETKPESKYVSLITSYQPFSLEKEVVCEEPLSPATVPGMHTEDN | |
| | PGKVEHTEELSSITEVVTTEENIPDVVPGSHLTPIERESSSPLSSNQ | |
| | SEPGSIALNSYHSRNCSESDHSRNGFDTDSSCLESHSSLSDSEFPPN | |
| | NKGEIKTEGQELITVIKAPTSFGYDKPHVLVDLLVDDSGKESLIGYR | |
| | PTEDSKEFS | |
| Interferon | MNPIVYWEYQIMPQVPVFTVEVKNYGVKNSEWIDACINISHHYCNIS | 61 |
| gamma | DHVGDPSNSLWVRVKARVGQKESAYAKSEEFAVCRDGKIGPPKLDIR | |
| receptor 1 | KEEKQIMIDIFHPSVFVNGDEQEVDYDPETTCYIRVYNVYVRMNGSE | |
| transcript | IQYKILTQKEDDCDEIQCQLAIPVSSLNSQYCVSAEGVLHVWGVTTE | |
| variant 10 | KSKEVCITIFNSSIKGSLWIPVVAALLLFLVLSLVFICFYIKKINPL | |
| | KEKSIILPKSLISVVRSATLETKPESKYVSLITSYQPFSLEKEVVCE | |
| | EPLSPATVPGMHTEDNPGKVEHTEELSSITEVVTTEENIPDVVPGSH | |
| | LTPIERESSSPLSSNQSEPGSIALNSYHSRNCSESDHSRNGFDTDSS | |
| | CLESHSSLSDSEFPPNNKGEIKTEGQELITVIKAPTSFGYDKPHVLV | |
| | DLLVDDSGKESLIGYRPTEDSKEFS | |
| Interferon | MNPIVYWEYQIMPQVPVFTVEVKNYGVKNSEWIDACINISHHYCNIS | 62 |
| gamma | DHVGDPSNSLWVRVKARVGQKESAYAKSEEFAVCRDGKIGPPKLDIR | |
| receptor 1 | KEEKQIMIDIFHPSVFVNGDEQEVDYDPETTCYIRVYNVYVRMNGSE | |
| transcript | IQYKILTQKEDDCDEIQCQLAIPVSSLNSQYCVSAEGVLHVWGVTTE | |
| variant 11 | KSKEVCITIFNSSIKGSLWIPVVAALLLFLVLSLVFICFYIKKINPL | |
| | KEKSIILPKSLISVVRSATLETKPESKYVSLITSYQPFSLEKEVVCE | |
| | EPLSPATVPGMHTEDNPGKVEHTEELSSITEVVTTEENIPDVVPGSH | |
| | LTPIERESSSPLSSNQSEPGSIALNSYHSRNCSESDHSRNGFDTDSS | |
| | CLESHSSLSDSEFPPNNKGEIKTEGQELITVIKAPTSFGYDKPHVLV | |
| | DLLVDDSGKESLIGYRPTEDSKEFS | |
| Interferon | MALLFLLPLVMQGVSRAEMGTADLGPSSVPTPTNVTIESYNMNPIVY | 63 |
| gamma | WEYQIMPQVPVFTVEVKNYGVKNSEWIDACINISHHYCNISDHVGDP | |
| receptor 1 | SNSLWVRVKARVGQKESAYAKSEEFAVCRDGKIGPPKLDIRKEEKQI | |
| transcript | MIDIFHPSVFVNGDEQEVDYDPETTCYIRVYNVYVRMNGSEIQYKIL | |
| variant 12 | TQKEDDCDEIQCQLAIPVSSLNSQYCVSAEGVLHVWGVTTEKSKEVC | |
| | ITIFNSSIKDLCGKKCYFRDKT | |
| Interferon | MALLFLLPLVMQGVSRAEMGTADLGPSSVPTPTNVTIESYNMNPIVY | 64 |
| gamma | WEYQIMPQVPVFTVEVKNYGVKNSEWIDACINISHHYCNISDHVGDP | |
| receptor 1 | SNSLWVRVKARVGQKESAYAKSEEFAVCRDGKIGPPKLDIRKEEKQI | |
| transcript | MIDIFHPSVFVNGDEQEVDYDPETTCYIRVYNVYVRMNGSEIQYKIL | |
| variant 13 | TQKEDDCDEIQCQLAIPVSSLNSQYCVSAEGVLHVWGVTTEKSKEIS | |
| | VVRSATLETKPESKYVSLITSYQPFSLEKEVVCEEPLSPATVPGMHT | |
| | EDNPGKVEHTEELSSITEVVTTEENIPDVVPGSHLTPIERESSSPLS | |
| | SNQSEPGSIALNSYHSRNCSESDHSRNGFDTDSSCLESHSSLSDSEF | |
| | PPNNKGEIKTEGQELITVIKAPTSFGYDKPHVLVDLLVDDSGKESLI | |
| | GYRPTEDSKEFS | |
| Interferon | MASVFRLYSAVPTPTNVTIESYNMNPIVYWEYQIMPQVPVFTVEVKN | 65 |
| gamma | YGVKNSEWIDACINISHHYCNISDHVGDPSNSLWVRVKARVGQKESA | |
| receptor 1 | YAKSEEFAVCRDGKIGPPKLDIRKEEKQIMIDIFHPSVFVNGDEQEV | |
| transcript | DYDPETTCYIRVYNVYVRMNGSEIQYKILTQKEDDCDEIQCQLAIPV | |
| variant 14 | SSLNSQYCVSAEGVLHVWGVTTEKSKEVCITIFNSSIKGSLWIPVVA | |
| | ALLLFLVLSLVFICFYIKKINPLKEKSIILPKSLISVVRSATLETKP | |
| | ESKYVSLITSYQPFSLEKEVVCEEPLSPATVPGMHTEDNPGKVEHTE | |
| | ELSSITEVVTTEENIPDVVPGSHLTPIERESSSPLSSNQSEPGSIAL | |
| | NSYHSRNCSESDHSRNGFDTDSSCLESHSSLSDSEFPPNNKGEIKTE | |
| | GQELITVIKAPTSF | |
| Interferon | MPTPTNVTIESYNMNPIVYWEYQIMPQVPVFTVEVKNYGVKNSEWID | 66 |
| gamma | ACINISHHYCNISDHVGDPSNSLWVRVKARVGQKESAYAKSEEFAVC | |
| receptor 1 | RDGKIGPPKLDIRKEEKQIMIDIFHPSVFVNGDEQEVDYDPETTCYI | |
| transcript | RVYNVYVRMNGSEIQYKILTQKEDDCD | |
| variant 15 | | |
| Interferon | MRPTLLWSLLLLLGVFAAAAAAPPDPLSQLPAPQHPKIRLYNAEQVL | 67 |
| gamma | SWEPVALSNSTRPVVYQVQFKYTDSKWFTADIMSIGVNCTQITATEC | |
| receptor 2 | DFTAASPSAGFPMDFNVTLRLRAELGALHSAWVTMPWFQHYRNVTVG | |
| transcript | PPENIEVTPGEGSLIIRFSSPFDIADTSTAFFCYYVHYWEKGGIQQV | |
| variant 1 | KGPFRSNSISLDNLKPSRVYCLQVQAQLLWNKSNIFRVGHLSNISCY | |
| | ETMADASTELQQVILISVGTFSLLSVLAGACFFLVLKYRGLIKYWFH | |
| | TPPSIPLQIEEYLKDPTQPILEALDKDSSPKDDVWDSVSIISFPEKE | |
| | QEDVLQTL | |
| Interferon | MRPTLLWSLLLLLGVFAAAAAAPPAQLTLETYQEWCNDSAATHDPLS | 68 |
| gamma | QLPAPQHPKIRLYNAEQVLSWEPVALSNSTRPVVYQVQFKYTDSKWF | |
| receptor 2 | TADIMSIGVNCTQITATECDFTAASPSAGFPMDENVTLRLRAELGAL | |
| transcript | HSAWVTMPWFQHYRNVTVGPPENIEVTPGEGSLIIRFSSPFDIADTS | |
| variant 2 | TAFFCYYVHYWEKGGIQQVKGPFRSNSISLDNLKPSRVYCLQVQAQL | |
| | LWNKSNIFRVGHLSNISCYETMADASTELQQVILISVGTFSLLSVLA | |
| | GACFFLVLKYRGLIKYWFHTPPSIPLQIEEYLKDPTQPILEALDKDS | |
| | SPKDDVWDSVSIISFPEKEQEDVLQTL | |
| Interferon | MSIGVNCTQITATECDFTAASPSAGFPMDENVTLRLRAELGALHSAW | 69 |
| gamma | VTMPWFQHYRNVTVGPPENIEVTPGEGSLIIRFSSPFDIADTSTAFF | |
| receptor 2 | CYYVHYWEKGGIQQVKGPERSNSISLDNLKPSRVYCLQVQAQLLWNK | |
| transcript | SNIFRVGHLSNISCYETMADASTELQQVILISVGTFSLLSVLAGACE | |
| variant 3 | FLVLKYRGLIKYWFHTPPSIPLQIEEYLKDPTQPILEALDKDSSPKD | |
| | DVWDSVSIISFPEKEQEDVLQTL | |
| Interferon | PKIRLYNAEQVLSWEPVALSNSTRPVVYQVQFKYTDSKWFTADIMSI | 70 |
| gamma | GVNCTQITATECDFTAASPSAGFPMDENVTLRLRAELGALHSAWVTM | |
| receptor 2 | PWFQHYRNVETAFLHVGQTGLELLTSSDLPTSASQSAGITVTVGPPE | |
| transcript | NIEVTPGEGSLIIRFSSPFDIADTSTAFFCYYVHYWEKGGIQQVKGP | |
| variant 4 | FRSNSISLDNLKPSRVYCLQVQAQLLWNKSNIFRVGHLSNISCYETM | |
| | ADASTELQQVILISVGTFSLLSVLAGACFFLVLKYRGLIKYWFHTPP | |
| | SIPLQIEEYLKDPTQPILEALDKDSSPKDDVWDSVSIISFPEKEQED | |
| | VLQTL | |
| Interferon | MAGPERWGPLLLCLLQAAPGRPRLAPPQNVTLLSQNFSVYLTWLPGL | 71 |
| lambda | GNPQDVTYFVAYQSSPTRRRWREVEECAGTKELLCSMMCLKKQDLYN | |
| receptor 1 | KFKGRVRTVSPSSKSPWVESEYLDYLFEVEPAPPVLVLTQTEEILSA | |
| transcript | NATYQLPPCMPPLDLKYEVAFWKEGAGNKTLFPVTPHGQPVQITLQP | |
| variant 1 | AASEHHCLSARTIYTFSVPKYSKFSKPTCFLLEVPEANWAFLVLPSL | |
| | LILLLVIAAGGVIWKTLMGNPWFQRAKMPRALDFSGHTHPVATFQPS | |
| | RPESVNDLFLCPQKELTRGVRPTPRVRAPATQQTRWKKDLAEDEEEE | |
| | DEEDTEDGVSFQPYIEPPSFLGQEHQAPGHSEAGGVDSGRPRAPLVP | |
| | SEGSSAWDSSDRSWASTVDSSWDRAGSSGYLAEKGPGQGPGGDGHQE | |
| | SLPPPEFSKDSGFLEELPEDNLSSWATWGTLPPEPNLVPGGPPVSLQ | |
| | TLTFCWESSPEEEEEARESEIEDSDAGSWGAESTQRTEDRGRTLGHY | |
| | MAR | |
| Interferon | MAGPERWGPLLLCLLQAAPGRPRLAPPQNVTLLSQNFSVYLTWLPGL | 72 |
| lambda | GNPQDVTYFVAYQSSPTRRRWREVEECAGTKELLCSMMCLKKQDLYN | |
| receptor 1 | KFKGRVRTVSPSSKSPWVESEYLDYLFEVEPAPPVLVLTQTEEILSA | |
| transcript | NATYQLPPCMPPLDLKYEVAFWKEGAGNKTLFPVTPHGQPVQITLQP | |
| variant 2 | AASEHHCLSARTIYTFSVPKYSKFSKPTCFLLEVPGLFWTHTPCGNL | |
| | SAQQTRVRE | |
| Interferon | MAGPERWGPLLLCLLQAAPGRPRLAPPQNVTLLSQNFSVYLTWLPGL | 73 |
| lambda | GNPQDVTYFVAYQSSPTRRRWREVEECAGTKELLCSMMCLKKQDLYN | |
| receptor 1 | KFKGRVRTVSPSSKSPWVESEYLDYLFEVEPAPPVLVLTQTEEILSA | |
| transcript | NATYQLPPCMPPLDLKYEVAFWKEGAGNKVGSSFPAPRLGPLLHPFL | |
| variant 3 | LRFFSPSQPAPAPLLQEVFPVHS | |
| Interferon | MMCLKKQDLYNKFKGRVRTVSPSSKSPWVESEYLDYLFEVEPAPPVL | 74 |
| lambda | VLTQTEEILSANATYQLPPCMPPLDLKYEVAFWKEGAGNKTLFPVTP | |
| receptor 1 | HGQPVQITLQPAASEHHCLSARTIYTFSVPKYSKFSKPTCFLLEVPE | |
| transcript | ANWAFLVLPSLLILLLVIAAGGVIWKTLMGNPWFQRAKMPRALV | |
| variant 4 | | |
| Interferon | MAGPERWGPLLLCLLQAAPGRPRLAPPQNVTLLSQNFSVYLTWLPGL | 75 |
| lambda | GNPQDVTYFVAYQSSPTRRRWREVEECAGTKELLCSMMCLKKQDLYN | |
| receptor 1 | KFKGRVRTVSPSSKSPWVESEYLDYLFEVEPAPPVLVLTQTEEILSA | |
| transcript | NATYQLPPCMPPLDLKYEVAFWKEGAGNKTLFPVTPHGQPVQITLQP | |
| variant 5 | AASEHHCLSARTIYTFSVPKYSKFSKPTCFLLEVPEANWAFLVLPSL | |
| | LILLLVIAAGGVIWKTLMGNPWFQRAKMPRALELTRGVRPTPRVRAP | |
| | ATQQTRWKKDLAEDEEEEDEEDTEDGVSFQPYIEPPSFLGQEHQAPG | |
| | HSEAGGVDSGRPRAPLVPSEGSSAWDSSDRSWASTVDSSWDRAGSSG | |
| | YLAEKGPGQGPGGDGHQESLPPPEFSKDSGFLEELPEDNLSSWATWG | |
| | TLPPEPNLVPGGPPVSLQTLTFCWESSPEEEEEARESEIEDSDAGSW | |
| | GAESTQRTEDRGRTLGHYMAR | |

Methods for determining or mapping the specific epitopes or nucleic acid sequences of epitopes that an antibody or autoantibody specifically binds are known in the art.

Examples of protocols for epitope mapping are described in Zuber et al., 2016 (Bartek Zuber, Karin Rudstrom, Cecilia Ehrnfelt, and Niklas Ahlborg.Epitope Mapping of Neutralizing Monoclonal Antibodies to Human Interferon-γ Using Human-Bovine Interferon-γ Chimeras.Journal of Interferon & Cytokine Research.September 2016.542-551.http://doi.org/10.1089/jir.2016.0017). To assess whether monoclonal antibodies (mAbs) or chimeric antibodies competed for simultaneous binding to IFN-γ, each mAb was used as a capture mAb and evaluated for binding to biotinylated E. coli-derived hIFN-γ in the presence of competitive mAbs First, to establish a suitable concentration of biotinylated hIFN-γ to use for each capture mAb, mAbs were coated and incubated with a serial dilution of hIFN-γ-biotin followed by detection with SA-HRP. The concentration of hIFN-γ-biotin yielding 50% of the maximal absorbance value (IC50) for each mAb was later used in the competition ELISA; the IC50 value divided by the molecular weight of hIFN-γ also yields the affinity constant (KD) at steady state for the mAbs. For the competitive ELISA, hIFN-γ-biotin, at the defined concentrations, was preincubated for 30 min with the mAbs to be tested for competition before being added to ELISA plates coated with capture mAb. After incubation, bound IFN-γ-biotin was detected using SA-HRP. The percentage inhibition achieved by a competitor mAb was calculated by comparing absorbance values to hIFN-γ-biotin incubated in the absence of competitor mAb.

Other examples of protocols for epitope mapping are described in Bastard et al., 2020, (Bastard e al., SCIENCE, 24 Sep. 2020, Vol 370, Issue 6515, DOI: 10.1126/science.abd4585) and Nolte et al., 1996 (Epitopes recognized by neutralizing therapy-induced human anti-interferon-α antibodies are localized within the N-terminal functional domain of recombinant interferon-α2, Nolte, Günther, von Wussow, September 1996, https://doi.org/10.1002/eji.1830260929).

A further of a protocol for epitope mapping is described by Novick et al., 2000 (Novick, Daniela; Nabioullin, Roustem R; Ragsdale, Wilbert; McKenna, Sean; Weiser, Weishui; et al., Journal of Interferon & Cytokine Research; New Rochelle Bd. 20, Ausg. 11, (November 2000): 971-82., DOI: 10.1089/10799900050198417). Therein a panel of monoclonal antibodies (mAb) derived against human interferon-a/b receptor-2 (IFNAR-2) was evaluated for their ability to antagonize the biologic effects of type 1 interferons (IFN-a1, IFN-a2a, and IFNb). Anti-IFNAR-2 mAb 117.7, 35.9, 53.2, and 51.44 neutralized type I IFN-mediated antiviral, antiproliferative, and major histocompatibility complex (MHC) class I upregulation functions.

Another protocol is disclosed in A. Chuntharapai, et al., 1999 (Determination of Residues Involved in Ligand Binding and Signal Transmission in the Human IFN-α Receptor 2, A. Chuntharapai, V. Gibbs, J. Lu, A. Ow, S. Marsters, A. Ashkenazi, A. De Vos and K. Jin Kim, J Immunol Jul. 15, 1999, 163 (2) 766-773). Therein Microtiter plates were coated with goat Abs specific for the Fc portion of human IgG in PBS and blocked with BSA. hIFNAR2-IgG (or hIFNAR2-IgG mutant) was added to each well and incubated with anti-hIFNAR2 mAbs (or hIFNAR2-IgG mutants). Wells were then incubated with HRP goat anti-mouse IgG. The bound enzyme was detected by the addition of TMB (3,3′,5,5′-tetramethylbenzidin) substrate, the reaction was stopped by the addition of stop solution, and the plates were read with an ELISA plate reader. Between each step, plates were washed three times in wash buffer. Epitope mapping using a competitive binding ELISA to determine whether mAbs recognized the same or different epitopes, a competitive binding ELISA was performed using biotinylated mAbs. mAbs were biotinylated using N-hydroxyl succinimide. The equilibrium dissociation and association constant rates of anti-hIFNAR2 mAbs were determined using an automated immunoassay system, as described, with a modification. Briefly, anti-human IgG agarose beads were coated with hIFNAR2-IgG, nonspecific binding sites were blocked. For equilibrium measurements, mAbs were mixed with a serial dilution of hIFNAR2-IgG and incubated. Once equilibrium was reached, this mixture was drawn through the beads. The anti-IFNAR2 mAbs bound to beads were detected by goat anti-mouse IgG. The equilibrium constant was calculated using the software provided by the manufacturer (Sapidyne).

In another aspect the present invention relates to the bi-specific agent according to the invention for use in the treatment of an (preferably anti-viral) immunodeficiency in a patient, wherein the patient is at least 60 years of age or older, and wherein the (preferably anti-viral) immunodeficiency in the patient is associated with the presence of one or more autoantibodies that bind an epitope of an interferon, an interferon receptor or combination thereof.

In embodiments, of the use for the treatment of an (preferably anti-viral) immunodeficiency in a patient, the patient is at least 60 years of age or older, and the (preferably anti-viral) immunodeficiency in the patient is associated with the presence of one or more autoantibodies that bind an epitope of an interferon, an interferon receptor or combination thereof, and/or wherein the patient is or has been treated with ((recombinant) human) interferon, e.g., IFNa2b, for another reason (e.g., due to a different condition/disease) and an autoimmunity (preferably comprising the production/excretion of autoantibodies) and/or a presence of one or more (e.g., anti-interferon) autoantibodies in the patient results from/is developed as a consequence (e.g., as side effect) of the interferon, e.g., IFNa2b, treatment, wherein in embodiments the interferon, e.g., IFNa2b, treatment has become ineffective or less effective in said patient (e.g., preferably due to the presence of (anti-interferon) autoantibodies in the patient).

In embodiments, the patient is or has been treated with ((recombinant) human) interferon for another reason/condition/disease, and a presence of one or more autoantibodies that bind an epitope of an interferon results from/is developed as a consequence or side effect of said interferon treatment in the patient, and preferably wherein the interferon treatment has become ineffective or less effective (e.g., shows reduced or no efficacy) in said patient.

In some of said embodiments a patient is or has been receiving an interferon treatment against another disease or condition (other than immunodeficiency), and has developed autoantibodies and/or an autoimmunity against interferon(s), or even against interferon receptor(s) or a combination thereof. In some of said embodiments the interferon treatment may even (have) become less effective or ineffective in said patient, for example, due to the presence of said autoantibodies. In embodiments, in such patients the restoration of interferon treatment efficacy and/or the treatment or prevention of an (anti-viral) immunodeficiency, preferably by the bi-specific agent according to the invention, may be advantageous.

As mentioned above, although addressing viral infection is a preferred embodiment of the invention, any pathogen infection in a subject may be worsened by a weakened immune response caused by anti-interferon or anti-interferon receptor autoantibodies. Thus, the approach described in the present invention is applicable towards treating and/or preventing any given infection in a subject, wherein viral infections represent a preferred embodiment. The relevant medical condition for treatment is thus any disorder in which an IFN response plays a role, for example any infection to which an IFN-mediated immune response provides a beneficial (anti-infective) effect.

Examples of conditions that may be addressed by the present invention include those associated with host antibody production against antigen(s) comprised in interferons and/or interferon receptors and which are preferably accompanied, characterized or associated with an impaired immunity against viral infection, such as anti-viral immunodeficiency.

In preferred embodiments the binding region is able to bind to an antibody released from a plasma cell, such that the plasma cell is then targeted by the immune system for destruction. This interaction between an antibody from a plasma cell and the binding region may be tested or confirmed, for example, in vitro, in vivo or ex vivo as disclosed herein to determine suitable bi-specific agents and optionally doses of bi-specific agent suitable for inducing the depletion and/or killing of plasma cells. The person skilled in the art is aware of other methods to test the specificity of the binding of an antibody to target antigens/epitopes.

In embodiments, of the use of the bi-specific agent according to the invention the interferon epitope of the binding region of the bi-specific agent is bound by one or more autoantibodies identified in the patient using an in vitro assay.

In embodiments of the use of the bi-specific agent according to the invention the in vitro assay comprises:
bringing a sample of the patient comprising antibodies into contact with at least one epitope of an interferon, an interferon receptor or a combination thereof, and
determining the presence of autoantibodies from the patient sample binding to said epitope and optionally determining the epitope bound by said autoantibodies,
wherein preferably the patient sample is a liquid biopsy, a blood or a serum sample,
wherein preferably the in vitro assay comprises:
   a competition binding assay using one or more additional antibodies that bind to known epitopes of an interferon, an interferon receptor or a combination thereof, or
   a binding assay using an oligopeptide array comprising multiple epitopes of an interferon, an interferon receptor or a combination thereof.

In embodiments of the use of the bi-specific agent according to the invention the treatment comprises cytotoxicity against plasma cells producing autoantibodies that bind an interferon, an interferon receptor or a combination thereof, preferably by antibody-dependent cellular cytotoxicity (ADCC) or complement dependent cytotoxicity (CDC).

Two non-limiting exemplary examples of in vitro assays are depicted in FIG. 1, were either competition binding assays with three-dimensional epitopes and (dye-labelled) antibodies with known binding specificities/target epitopes are used to determine the presence and specificity of autoantibodies present in a patient sample (v1). The other non-limiting example depicted in FIG. 1 (v2) is the use of binding assays or competitive binding assays using two-dimensional or linear epitopes, which may be immobilized, e.g., on a surface or array.

Once the specificity or target epitope of an autoantibody in a patient is known, suitable bi-specific agents may be used to target the respective autoantibody-producing cells in a patient or patient sample. Non-limiting exemplary embodiments of such (therapeutic) bi-specific agents are depicted on the right side of FIG. 1, where a bi-specific agent my either comprise one or more three-dimensional target epitopes of an autoantibody or one or more linear or two-dimensional epitopes, preferably in its binding region. The anchor region of said bi-specific agents depicted exemplary in FIG. 1 is directed towards a plasma cell, which supposedly produces and/or secretes said autoantibody of interest. The treatment of a patient with the bi-specific agent according to the invention preferably induces cell death in said plasma cells that are bound by the bi-specific agent, such that the amount of secreted autoantibodies in a patient is reduced, and the anti-viral immunity or immunocompetence is preferably restored or improved.

Other assays for mapping or determining the target epitope of an antibody or autoantibodies present in a sample are known in the art.

The bi-specific agent according to the present invention may be administered at a daily dosage of from about 0.005 mg to about 100 mg per kg of body weight of the individual, for example given as a single daily dose, or a once weekly dose, or a dose every 2 weeks, three weeks, 1 month, 2 months or 3 months, or more. This dosage regimen may be adjusted to provide the optimal therapeutic response. Suitable individual doses of a bi-specific agent, eg., in form of a polypeptide may include 10-1000 mg, such as 10-100 mg, 100-200 mg, 200-300 mg, 300-400 mg, 400-500 mg, and 500-1000 mg.

It will be understood that the specific dose level and frequency of dosage for any particular patient may be varied and will depend upon a variety of factors including the activity of the specific compound (bi-specific agent) employed, the metabolic stability and length of action of that compound, the age, body weight, general health, sex, diet, mode and time of administration, rate of excretion, drug combination, the severity of the particular condition, and the host undergoing therapy. Generally, where the bi-specific agent is a polypeptide, then the amount of antigen in each dose of bi-specific agent is selected as an amount which is effective to produce cell killing of cells that are bound by the anchor region without significant, adverse side effects in typical subjects. Such amount may vary depending on which specific polypeptides are employed. An optimal amount for a particular bi-specific agent can be ascertained by standard studies involving observation of antibody titers and other responses in subjects.

In another aspect the present invention relates to an in vitro assay for identifying autoantibodies that bind an epitope of an interferon, an interferon receptor or combination thereof, comprising:
bringing into contact a sample of a patient comprising antibodies with at least one epitope of an interferon, an interferon receptor or combination thereof, and
determining the presence of autoantibodies from the patient sample binding to said epitope,
wherein the patient sample is preferably a liquid biopsy, a blood or a serum sample, and
wherein the epitope is preferably linked to a solid phase.

In embodiments, the in vitro assay according to the invention further comprises bringing into contact the sample of a patient with multiple epitopes of an interferon, an interferon receptor or combination thereof, and determining the epitope bound by said autoantibodies.

In embodiments, the in vitro assay according to the invention additionally comprises providing a pharmaceutical composition comprising a bi-specific agent according to the invention, wherein the agent comprises, as the binding region, an epitope of an interferon, an interferon receptor or combination thereof, that was determined to be bound by said autoantibodies.

The invention thus relates to personalized medicine, in which the relevant interferon or interferon receptor epitope (antigen) of the binding region of the bi-specific agent is selected based on autoantibodies identified in the subject requiring treatment. The invention further relates to a companion diagnostic, in which ongoing assessment of anti-interferon or anti-interferon receptor autoantibodies is carried out, to determine which epitopes should be employed in the therapeutic agent. Multiple assessments may be carried over time, including assessment prior to, during and/or after treatment, to determine if autoantibody titers are reduced and whether other autoantibodies are evident, that may require targeting by (another) bis-specific agent of the invention with the relevant autoantigen (autoepitope) in the binding region.

In embodiments, the ability to directly identify a plasma cell secreting a specific (auto-) antibody of interest, e.g., directed against an epitope comprised in an interferon and/or interferon receptior, can be used to select such plasma cells as well as to eliminate these cells.

In another aspect the present invention relates to a method for the in vitro elimination of a plasma cell secreting an autoantibody that binds an epitope of an interferon, an interferon receptor or a combination thereof, the method comprising:
contacting a sample from a patient comprising a plasma cell secreting at least one autoantibody that binds an epitope of an interferon, an interferon receptor or a combination thereof, with a bi-specific agent according to the invention or a nucleic acid according to the invention.

In vitro, the nature of the interaction between a binding region and an (auto-) antibody can be probed by contacting a plasma cell with the bi-specific agent according to the invention and then providing that plasma cell with an antibody directed to the binding region in order to determine the degree of interaction of that antibody with binding region, for example, by monitoring the extent of cell killing as a surrogate for the binding efficacy. The efficacy of the antibody-binding region interaction can therefore be probed in vitro, allowing optimization of the interaction, if needed.

Therefore, in one further aspect the invention provides a method or use as disclosed herein, the method or use comprising delivering to an individual, or cells, or tissue, a composition of the invention and then further delivering to the individual, cells or tissue respectively, an antibody capable of a specific interaction with the binding region of the composition, optionally followed by measurement of cell killing. The antibody may be provided in any suitable amount to facilitate cell killing, for example.

In another aspect the present invention relates to a method of treating an anti-viral immunodeficiency in a patient, the method comprising:
administering to a patient a pharmaceutically effective amount of a bi-specific agent according to the invention, preferably by oral, intravenous, intramuscular, parenteral, subcutaneous, transdermal, epidural administration or inhalation, wherein the patient has been positively tested for producing autoantibod(y)ies specific to at least one epitope of an interferon, an interferon receptor or a combination thereof, and wherein the treatment preferably induces the in vivo elimination of autoantibody-secreting plasma cells, wherein said autoantibodies bind to at least one epitope of an interferon, an interferon receptor or a combination thereof.

In another aspect the present invention relates to a kit for performing the in vitro assay according to the invention, comprising a bi-specific agent according to the invention, optionally linked to a solid phase, or a nucleic acid molecule according to the invention.

The invention further relates to methods using the bi-specific agent according to the invention, for example, to target, kill and/or deplete plasma cells expressing (auto-)antibodies targeting epitope(s) comprised in interferons and/or interferon receptors and that are associated with anti-viral immuncompromization of a patient.

Hence, the invention provides a method for prevention and/or treatment of a medical condition caused or exacerbated by an antibody released by a plasma cell, the method comprising delivery to an individual a bi-specific agent as disclosed herein.

A bi-specific agent as disclosed herein for use in for prevention and/or treatment of a medical condition caused or exacerbated by an antibody released by a plasma cell.

Use of a bi-specific agent as disclosed herein in the preparation of a medicament for the prevention and/or treatment of a medical condition caused or exacerbated by an antibody released by a plasma cell.

In embodiments, the bi-specific agent is delivered to an individual in need thereof in an effective amount to reduce antibody production from the plasma cell to which the bi-specific agent is bound. Said plasma cell secreting and/or producing antibodies targeting at least one epitope comprised in an interferon, an interferon receptor or a combination thereof.

In embodiments, the bi-specific agent disclosed herein is delivered to an individual in need thereof in combination with a medicament capable of preventing the regeneration of plasma cells, which may be selected from, for example, an anti-CD20 antibody, such as rituximab, ocrelizumab, ofatumumab, or drugs able to target BLyS and APRIL (for example anti-BLyS such as Belivumab, and TACI-Ig such as Atacicept respectively). Preferably such combination treatment would serve to kill or deplete specific plasma cells producing antibodies associated with the medical condition of immuncompromization described herein, as well as prevent the development of new plasma cells generating the same antibody.

In embodiments, the invention relates to a bi-specific agent or polynucleic acid encoding such a bi-specific agent provided in combination with a medicament capable of preventing the regeneration of plasma cells, optionally wherein the medicament is selected from an anti CD20 antibody such as rituximab, ocrelizumab or ofatumumab or drugs able to target BLyS and APRIL (for example anti-BLyS such as Belivumab, and TACI-Ig such as Atacicept respectively). The two agents may be administered simultaneously, substantially simultaneously or sequentially.

In another aspect the methods and uses of the invention comprise treating an individual by plasmapheresis or other methods known to reduce or eliminate serum antibodies prior to delivery of the bi-specific agent according to the invention. Hence, the invention relates to a method or bi-specific agent as disclosed herein, wherein the individual to receive a treatment according to the invention has been previously subjected to a plasmapheresis treatment to reduce or eliminate serum antibodies.

In another aspect the invention provides a method for killing an antibody-producing cell in vivo, in vitro or ex vivo comprising contacting, with a bi-specific agent according to the invention, a sample containing a plasma cell, the bi-specific agent being in an amount effective to result in plasma cell killing. Wherein the plasma cell targeted by the bi-specific agent secretes and/or produces antibodies targeting at least one epitope comprised in an interferon, an interferon receptor or a combination thereof. The sample may be a liquid such as blood or plasma, or a solid such as a tissue or part thereof. For example, blood or plasma may be removed from a body and once removed, treated by the method according to the invention.

In one embodiment the bi-specific agent according to the invention is provided in an environment capable of effecting complement lysis and/or antibody-dependent cellular cytotoxicity (ADCC).

The invention further relates to the use of a bi-specific agent according to the invention in the elimination in vivo of specific antibody producing cells, wherein said antibod(y)ies target at least one epitope comprised in an interferon, an interferon receptor or a combination thereof, even where such cells and the antibodies they produce are not associated with a medical condition, such as immuncompromization. For example, bi-specific agents according to the invention may be for use in animal models, for example in situations in which it is desired to remove or reduce plasma cells and the antibodies they produce for reasons unrelated to a medical condition, but which are advantageous for the use of the animal model.

Embodiments and features of the invention described with respect to the bi-specific agent, the in vivo methods and kits are considered to be disclosed with respect to each and every other aspect of the disclosure, such that features characterizing the bi-specific agent, may be employed to characterize the methods, or kit and vice-versa. The various aspects of the invention are unified by, benefit from, are based on and/or are linked by the common and surprising finding of the depletion of plasma cells producing anti-interferon and/or interferon receptor targeting autoantibodies through the delivery of a bi-specific agent comprising an anchor region capable of binding a plasma cell, and a binding region associated with the anchor region, wherein the binding region comprises an epitope of an interferon, an interferon receptor or combination thereof, bound by one or more autoantibodies.

### DETAILED DESCRIPTION OF THE INVENTION

All cited documents of the patent and non-patent literature are hereby incorporated by reference in their entirety.

The invention is directed to a bi-specific agent, comprising an anchor region capable of binding a plasma cell, and a binding region associated with the anchor region, wherein the binding region comprises an epitope of an interferon, an interferon receptor or combination thereof, capable of binding one or more autoantibodies.

The invention further relates to the used of said a bi-specific agent, in the treatment of an anti-viral immunodeficiency in a patient, wherein the anti-viral immunodeficiency in the patient is associated with the presence of one or more autoantibodies that bind an epitope of an interferon, an interferon receptor or combination thereof.

Herein a “host” refers to a subject or patient which might suffer from pathogen, preferably viral, infections and/or preferably is suspected or has been diagnosed be immunocompromised or to comprise an immune deficiency. Possible hosts of the present invention as a mammal, preferably a human.

The term “subject” or “patient” include or preferably refer to subjects of 60 years of age and older.

In embodiments, a subject or patient may be suspected or predicted to have an immunodeficiency, more specifically an anti-viral immuncompromization or immunodeficiency, which is suspected to be at least partially due to the presence of autoantibodies directed against at least one epitope comprised by an interferon or interferon receptor or a combination thereof. The terms “subject” or “patient” may be used herein interchangeably. Herein as subject, patient or host may be an organism, a cell culture of patient cells or cell lines, an animal, a model-animal, or a cell culture of animal cells or cell lines, preferably a human subject or patient.

Herein a “sample” may be taken from a patient, a cell culture of patient cells or cell lines, an animal, or a cell culture of animal cells or cell lines of a biopsy, a blood sample, a tissue sample, an environmental sample, or a food-derived sample. Basically, any kind of sample that is suspected to contain nucleic acid sequence of interest, such as target sequences, among other. As used herein, a sample is a biological sample that is obtained or isolated from the patient or subject. Sample, as used herein may, e.g., refer to a sample of bodily fluid or tissue obtained for the purpose of diagnosis, prognosis, or evaluation of a subject of interest, such as a patient. Preferably herein, the sample is a sample of a bodily fluid, such as blood, serum, plasma, cerebrospinal fluid, urine, saliva, sputum, pleural effusions, cells, a cellular extract, a tissue sample, a tissue biopsy, a stool sample and the like. Particularly, the sample is blood, blood plasma, or blood serum. A sample herein may be selected from the group comprising a liquid sample, a solid sample, a biopsy, a liquid biopsy, a tissue sample, a cell culture sample or a swap-derived sample. The sample may further comprise or be a bodily fluid, whole blood or blood parts, plasma, serum, cells, tissue, saliva, sputum, mucus, phlegm, semen, vaginal fluids, liquor or cerebrospinal fluid, urine or pleural effusions.

“Specific binding” is to be understood as via one skilled in the art, whereby the skilled person is clearly aware of various experimental procedures that can be used to test binding and binding specificity. Methods for determining equilibrium association or equilibrium dissociation constants are known in the art. Some cross-reaction or background binding may be inevitable in many protein-protein interactions; this is not to detract from the “specificity” of the binding between an antibody and epitope. By way of example, “specific binding” describes binding of an e.g. anti-interferon antibody or antigen binding fragment thereof to interferon at greater binding affinity than background binding. The term “directed against” is also applicable when considering the term “specificity” in understanding the interaction between antibody and epitope.

An “antigen (Ag)” refers to a compound, composition, or substance that can stimulate the production of antibodies in an animal. In particular embodiments, the target antigen is an epitope of a interferon, or interferon receptor or a combination thereof. An “epitope” refers to the region of an antigen to which a binding agent binds. Epitopes can be formed both from contiguous amino acids or noncontiguous amino acids juxtaposed by tertiary folding of a protein. Any suitable antigen can be selected depending on the protein or epitope that is targeted by autoantibodies of a patient, preferably any type of interferon or interferon receptor or any combination thereof.

“Single-chain Fv” or “scFv” antibody fragments comprise the VH and VL domains of an antibody, wherein these domains are present in a single polypeptide chain and in either orientation (e.g., VL-VH or VH-VL). Generally, the scFv polypeptide further comprises a polypeptide linker between the VH and VL domains which enables the scFv to form the desired structure for antigen binding. In preferred embodiments, an antibody contemplated herein comprises antigen-specific binding domain that is a scFv and may be a murine, human or humanized scFv. Single chain antibodies may be cloned from the V region genes of a hybridoma specific for a desired target.

In particular embodiments, the antigen-specific binding domain that is a humanized scFv that binds a polypeptide of a human interferon, or interferon receptor or any combination thereof.

Antibodies or antibody fragments of the invention therefore include, but are not limited to polyclonal, monoclonal, bispecific, human, humanized or chimeric antibodies, single chain fragments (scFv), single variable fragments (ssFv), single domain antibodies (such as VHH fragments from nanobodies), Fab fragments, F(ab′) 2 fragments, fragments produced by a Fab expression library, anti-idiotypic antibodies and epitope-binding fragments or combinations thereof of any of the above, provided that they retain similar binding properties of the antibodies described herein, preferably comprising the corresponding CDRs, or VH and VL regions as described herein. Also, mini-antibodies and multivalent antibodies such as diabodies, triabodies, tetravalent antibodies and peptabodies can be used in a method of the invention. The immunoglobulin molecules of the invention can be of any class (i.e. IgG, IgE, IgM, IgD and IgA) or subclass of immunoglobulin molecules.

As used herein, an “antibody” generally refers to a protein consisting of one or more polypeptides substantially encoded by immunoglobulin genes or fragments of immunoglobulin genes. Where the term “antibody” is used, the term “antibody fragment” may also be considered to be referred to. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. The basic immunoglobulin (antibody) structural unit is known to comprise a tetramer or dimer. Each tetramer is composed of two identical pairs of polypeptide chains, each pair having one “light” (L) (about 25 kD) and one “heavy” (H) chain (about 50-70 kD). The N-terminus of each chain defines a variable region of about 100 to 110 or more amino acids, primarily responsible for antigen recognition. The terms “variable light chain” and “variable heavy chain” refer to these variable regions of the light and heavy chains respectively. Optionally, the antibody or the immunological portion of the antibody, can be chemically conjugated to, or expressed as, a fusion protein with other proteins.

A variable region of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies.

A single-domain antibody, also called a ‘nanobody’, is an antibody fragment consisting of a single monomeric variable antibody domain. Like a complete antibody, nanobodies can selectively bind to an antigen. Nanobodies commonly have a molecular weight of only 12-15 kDa, and are significantly smaller than conventional antibodies (150-160 kDa), which are composed of two heavy and two light protein chains, and also smaller than single-chain variable fragments (about 25 kDa, two variable domains, comprising one from a light chain and one from a heavy chain) and Fab fragments (about 50 kDa, comprising one light and half a heavy chain). “Peptide” “polypeptide”, “polypeptide fragment” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring.

“Fusion proteins” or “chimeric proteins” as used herein, comprise proteins formed by joining two or more nucleic acid segments that originally encoded separate proteins, polypeptides or protein fragments. “Fusion gene”, “gene fusion” or “fused gene” are according to the invention preferably joined nucleic acid sequence segments, wherein at least one nucleic acid sequence is also an exogenous nucleic acid sequence and at least one other nucleic acid sequence is also an endogenous nucleic acid sequence. In preferred embodiments, the exogenous nucleic acid is integrated into and fused to an endogenous nucleic acid sequence, e.g., an endogenous gene. In some embodiments, the integrated exogenous nucleic acid sequence to be fused may have several combined gene structures and/or polypeptide-encoding sequences separated by one or more protein separation sites.

The translation of a fusion gene results in a single or multiple polypeptides with functional properties derived from each of the original proteins or protein fragments. In a further embodiment, a fused gene may contain one or more nucleic acid sequences encoding a polypeptide, wherein the polypeptides may be present as individual polypeptides or proteins after translation, with separation being mediated by a protein separation site.

Herein in embodiments the bi-specific agent or a fragment thereof can be provided or can constitute a chimeric protein, chimera or chimeras, wherein said term refers to hybrid proteins made up of polypeptides with different functions or attributes. In some embodiments, fusion proteins (e.g., the bi-specific agent or a fragment thereof) combine whole peptides and therefore contain all functional domains of the original proteins. In certain embodiments, fusion proteins combine only parts of coding sequences and therefore only retain parts of the original functions of the genes from which they were formed.

As used herein, the terms “polynucleotide” or “nucleic acid molecule” refers to messenger RNA (mRNA), RNA, genomic RNA (gRNA), plus strand RNA (RNA(+)), minus strand RNA (RNA(−)), genomic DNA (gDNA), complementary DNA (cDNA) or recombinant DNA. Polynucleotides include single and double stranded polynucleotides. Preferably, polynucleotides of the invention include polynucleotides or variants having at least about 50%, 55%, 60%, 65%, 70%, 75%, 80%, 85%, 90%, 95%, 96%, 97%, 98%, 99% or 100%) sequence identity to any of the reference sequences described herein, typically where the variant maintains at least one biological activity of the reference sequence.

In various illustrative embodiments, the present invention contemplates, in part, polynucleotides comprising expression vectors, viral vectors, and transfer plasmids, and compositions, and cells comprising the same. Polynucleotides can be prepared, manipulated and/or expressed using any of a variety of well-established techniques known and available in the art. In order to express a desired polypeptide, a nucleotide sequence encoding the polypeptide, can be inserted into appropriate vector. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or MI 3 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). In particular embodiments, the coding sequences of the chimeric proteins disclosed herein can be ligated into such expression vectors for the expression of the chimeric protein (e.g., the bi-specific agent or a fragment thereof) in mammalian cells. The “control elements” or “regulatory sequences” present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions-which interact with host cellular proteins to carry out transcription and translation. Such elements may vary in their strength and specificity. Depending on the vector system and host utilized, any number of suitable transcription and translation elements, including ubiquitous promoters and inducible promoters may be used.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

Sequence variants of the claimed nucleic acids, proteins, antibodies, antibody fragments and/or bi-specific agents, for example those defined by % sequence identity, that maintain similar binding properties of the invention are also included in the scope of the invention. Such variants, which show alternative sequences, but maintain essentially the same binding properties, such as target specificity, as the specific sequences provided are known as functional analogues, or as functionally analogous. Sequence identity relates to the percentage of identical nucleotides or amino acids when carrying out a sequence alignment. The recitation “sequence identity” as used herein refers to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

It will be appreciated by those of ordinary skill in the art that, as a result of the degeneracy of the genetic code, there are many nucleotide sequences that encode a polypeptide as described herein. Some of these polynucleotides bear minimal homology or sequence identity to the nucleotide sequence of any native gene. Nonetheless, polynucleotides that vary due to differences in codon usage are specifically contemplated by the present invention. Deletions, substitutions and other changes in sequence that fall under the described sequence identity are also encompassed in the invention.

Protein sequence modifications, which may occur through substitutions, are also included within the scope of the invention. Substitutions as defined herein are modifications made to the amino acid sequence of the protein, whereby one or more amino acids are replaced with the same number of (different) amino acids, producing a protein which contains a different amino acid sequence than the primary protein. Substitutions may be carried out that preferably do not significantly alter the function of the protein. Like additions, substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function.

In general, the non-polar amino acids Gly, Ala, Val, lie and Leu; the non-polar aromatic amino acids Phe, Trp and Tyr; the neutral polar amino acids Ser, Thr, Cys, Gin, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups. Substitution variants have at least one amino acid residue in the antibody molecule removed and a different residue inserted in its place. The sites of greatest interest for substitutional mutagenesis include the hypervariable regions, but framework alterations are also contemplated. If such substitutions result in a change in biological activity, then more substantial changes, denominated “exemplary substitutions” in the table immediately below, or as further described below in reference to amino acid classes, may be introduced and the products screened.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Conservative amino acid substitutions are not limited to naturally occurring amino acids, but also include synthetic amino acids. Commonly used synthetic amino acids are omega amino acids of various chain lengths and cyclohexyl alanine which are neutral non-polar analogues; citrulline and methionine sulfoxide which are neutral non-polar analogues, phenylglycine which is an aromatic neutral analog; cysteic acid which is a negatively charged analog and ornithine which is a positively charged amino acid analogue. Like the naturally occurring amino acids, this list is not exhaustive, but merely exemplary of the substitutions that are well known in the art.

For example, the sequences provided in Table 1, or the underlined sequences provided in Table 1, may, in embodiments, be used as the epitope of the binding region of the bi-specific agent. Variants of these sequences, as described for example in the passage above, may also, in embodiments, be employed as the binding region.

Variations in length of the epitope of Table 1 may also be employed. For example, in embodiments, the polypeptide may have, or the nucleic acid encodes a polypeptide that may have, a 0 to 10 amino acid addition or deletion at the N and/or C terminus of a sequence, with reference to the specific sequences provided herein. As used herein the term “a 0 to 10 amino acid addition or deletion at the N and/or C terminus of a sequence” means that the polypeptide may have a) 0, 1, 2, 3, 4, 5, 6, 7, 8, 9 or 10 additional amino acids at its N terminus and 0, 1, 2, 3, 4, 5, 6, 7, 8, 9 or 10 amino acids deleted at its C terminus or b) 0, 1, 2, 3, 4, 5, 6, 7, 8, 9 or 10 additional amino acids at its C terminus and 0, 1, 2, 3, 4, 5, 6, 7, 8, 9 or 10 nucleotides deleted at its N terminus, c) 0, 1, 2, 3, 4, 5, 6, 7, 8, 9 or 10 additional amino acids at its N terminus and 0, 1, 2, 3, 4, 5, 6, 7, 8, 9 or 10 additional amino acids at its N terminus or d) 0, 1, 2, 3, 4, 5, 6, 7, 8, 9 or 10 amino acids deleted at its N terminus and 0, 1, 2, 3, 4, 5, 6, 7, 8, 9 or 10 amino acids deleted at its C terminus.

Furthermore, in addition to the polypeptides described herein, peptidomimetics are also contemplated. Peptide analogues are commonly used in the pharmaceutical industry as non-peptide drugs with properties analogous to those of the template peptide. These types of non-peptide compound are termed “peptide mimetic” or “peptidomimetic” (Fauchere (1986) Adv. Drug Res. 15: 29; Veber and Freidinger (1985) TINS p. 392; and Evans et al. (1987) J. Med. Chem. 30:1229) and are usually developed with the aid of computerized molecular modelling. Peptide mimetics that are structurally similar to therapeutically useful peptides may be used to produce an equivalent therapeutic or prophylactic effect. It may be preferred in some embodiments to use peptide mimetics in order to prolong the stability of the polypeptides, when administered to a subject. To this end peptide mimetics for the polypeptides may be preferred.

Immunodeficiency, also known as immuncompromization, can be defined as a state in which the ability of the immune system of a subject is not capable or at least partially incapable of fighting infectious diseases or cancer. A person who has an immunodeficiency of any kind is considered to be immunocompromized. An immunocompromized individual may be particularly vulnerable to infections, such as viral infections. Adult-onset immunodeficiency might be caused by anti-interferon (e.g., interferon gamma) autoantibodies and is commonly associated with an increased susceptibility to viral infections or disseminated infections caused by opportunistic pathogens.

Interferons (IFNs) are a group of signaling proteins produced and released by a subjects cells in response to the presence of viruses. During viral infections the virus-infected cells releases interferons that cause neighboring cells to increase their anti-viral defenses. IFNs belong to the class of proteins called cytokines. Cytokines are messenger molecules that mediate the communication between cells and -in the case of interferons-trigger immune system defenses that help eradicate pathogens. Interferons are named for their ability to disrupt viral replication by protecting cells from viral infection. IFNs also have various other functions, such as the activation of immune cells, e.g., macrophages or natural killer cells, the potentiation of a hosts defense against pathogens through the upregulating of antigen presentation (through the increase of major histocompatibility complex (MHC) antigen-expression. More than twenty different interferon genes and proteins are known in humans, which are commonly divided into three classes, based on the type of receptor through which they signal as: Type I IFN, Type II IFN, and Type III IFN. Interferons of all classes are important for the immune systems defense against viral infections and also for the regulation of the immune system itself. Presently known human interferons comprise IFNA1, IFNA2, IFNA4, IFNA5, IFNA6, IFNA7, IFNA8, IFNA10, IFNA13, IFNA14, IFNA16, IFNA17, IFNA21, IFNB1, IFNW, IFNE1 and IFNK.

All Interferon Type I bind to a specific cell-surface receptor complex known as the IFN-α/β-receptor (IFNAR), composed of IFNAR1 and IFNAR2 chains. Human type I interferons are IFN-α, IFN-β, IFN-ε, IFN-κ, and IFN-ω. Type I interferons are produced by fibroblasts and monocytes as soon as the immune system recognizes a viral infection. The production of type I IFN-α can be inhibited by the cytokine interleukin-10. Once released, type I interferons bind to specific receptors on target cells, resulting in the expression of proteins that prevent the virus from producing and replicating its RNA and DNA.

Interferon Type II (IFN-γ in humans) binds to the receptor IFNGR, which consists of IFNGR1 and IFNGR2 chains. Type II IFNs are also released by cytotoxic T cells and type 1 helper T cells and represent “immune interferons”, which are activated by interleukin-12. Type II interferons are able to block the proliferation of type 2 T helper cells, which results in inhibition of Th2 immune response and further induction of Th1 immune response.

Type III interferons signal through a receptor complex consisting of IL10R2 (also called CRF2-4) and IFNLR1 (also called CRF2-12). Type III IFNs are important mediators of immune response in some types of viral or fungal infections.

The present invention encompasses both treatment and prophylactic treatment of a subject. A “prophylactic” treatment is a treatment administered to a subject who does not exhibit signs of a disease or exhibits only early signs for the purpose of decreasing the risk of developing pathology.

The bi-specific agent according to the invention may also be administered in a pharmaceutical composition prepared for administration to a subject and which comprise a therapeutically effective amount of one or more of the compounds disclosed herein. In embodiments, the pharmaceutical compositions are useful for treating or preventing an immunodeficiency related to the presence of autoantibodies targeting at least one epitope comprised in an interferon or interferon receptor or a combination thereof. The therapeutically effective amount of a bi-specific agent depends on the route of administration, the species of subject and the physical characteristics of the subject being treated. Specific factors that can be taken into account include weight, diet and concurrent medications of a subject. The relationship of these factors to determining a therapeutically effective amount of a bi-specific agent is understood by those of skill in the art.

The compositions contemplated herein may comprise one or more polypeptides, polynucleotides, vectors comprising same, genetically modified immune effector cells, etc., as contemplated herein. Compositions include, but are not limited to, pharmaceutical compositions.

Pharmaceutical compositions comprising a bi-specific agent for administration to a subject can include in embodiments at least one further pharmaceutically acceptable additive such as carriers, thickeners, diluents, buffers, preservatives, surface active agents and the like in addition to the molecule of choice. Pharmaceutical compositions can also include one or more additional active ingredients such as antimicrobial agents, anti-inflammatory agents, anesthetics, and the like. The pharmaceutically acceptable carriers useful for these formulations are conventional. The person skilled in the art is aware of compositions and formulations suitable for pharmaceutical delivery of the bi-specific agent disclosed herein. In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The bi-specific agent can in embodiments be combined with pharmaceutically acceptable carrier substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions comprising the bi-specific agent, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like. Liquid pharmaceutical compositions, whether they are solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

A bi-specific agent or a bi-specific agent comprised in a (pharmaceutical) composition can be administered to subjects by a variety of mucosal administration modes, including by oral, rectal, intraocular, intranasal, intrapulmonary, or transdermal delivery, intramuscular, intraocular, subcutaneous, intravenous, intra-arterial, intra-articular, intraperitoneal, intrathecal, intracerebroventricular, or parenteral routes.

In accordance with the disclosure herein, a prophylactically or therapeutically effective amount of the bi-specific agent may in embodiments be administered to a subject in need of such treatment for a time and under conditions sufficient to prevent, inhibit, and/or ameliorate a selected condition or one or more symptom(s) thereof, wherein the condition is associated with the presence of autoantibodies directed against at least one epitope comprised within an interferon, an interferon receptor or a combination thereof.

“Administration of and “administering” should be understood to mean providing a compound, namely the bi-specific agent, or a pharmaceutical composition comprising the bi-specific agent as described herein. The bi-specific agent or composition can be administered by another person to the subject (e.g., intravenously) or it can be self-administered by the subject (e.g., tablets).

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” or “prophylactic” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

Dosage can be varied by the attending clinician to maintain a desired concentration at a target site (for example, the lungs or systemic circulation). Higher or lower concentrations can be selected based on the mode of delivery, for example, trans-epidermal, rectal, oral, pulmonary, or intranasal delivery versus intravenous or subcutaneous delivery. Dosage can also be adjusted based on the release rate of the administered formulation, for example, of an intrapulmonary spray versus powder, sustained release oral versus injected particulate or transdermal delivery formulations, and so forth.

A “therapeutically effective amount” refers to a quantity of a specified agent, such as a bi-specific agent, sufficient to achieve a desired effect in a subject being treated with that agent. For example, this may be the amount of a bi-specific agent disclosed herein useful in treating a condition described herein in a subject. The therapeutically effective amount or diagnostically effective amount of a bi-specific agent will be dependent on the subject being treated, the severity of the affliction, and the manner of administration of the therapeutic composition. Dosage regimens can be adjusted to provide an optimum prophylactic or therapeutic response. A therapeutically effective amount is also one in which any toxic or detrimental side effects of the compound and/or other biologically active agent is outweighed in clinical terms by therapeutically beneficial effects. A non-limiting range for a therapeutically effective amount of a bi-specific agent and/or composition comprising the same within the methods and formulations of the disclosure is, e.g., about 0.001 mg/kg body weight to 50 mg/kg body weight, 0.01 mg/kg body weight to about 20 mg/kg body weight, such as about 0.05 mg kg to about 5 mg/kg body weight, or about 0.2 mg/kg to about 2 mg/kg body weight.

The instant disclosure also includes kits, packages and multi-container units containing the herein described bi-specific agent or pharmaceutical compositions comprising the same and/or means for administering the same for use in the prevention and treatment of conditions described herein and other conditions in human subjects.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, where relevant, the term “about” is used to indicate that a value includes the inherent variation of error for the device, the method being employed to determine the value, or the variation that exists among the study subjects.

### FIGURES

The invention is further described by the following figures. These are not intended to limit the scope of the invention but represent preferred embodiments of aspects of the invention provided for greater illustration of the invention described herein.

FIG. 1: Schematic depiction showing one embodiment of bi-specific agents according to the invention, which may serve as therapeutic proteins for the depletion of plasma cells secreting interferon- and/or interferon receptor specific autoantibodies. The left column of bi-specific agents comprises an anchor region capable of binding a plasma cell (“anti-plasma cell F-v”), and a binding region associated with the anchor region, wherein the binding region comprises an epitope of an interferon, an interferon receptor or combination thereof, bound by one or more autoantibodies present in the patient sample, wherein said epitope may be a two-dimensional or linear epitope (L1-L4 epitope). On the fare most right of the scheme a bi-specific agent comprising an anchor region capable of binding a plasma cell (“anti-plasma cell F-v”), and a binding region associated with the anchor region, wherein the binding region comprises a “holoantigen” comprising several three-dimensional epitopes of an interferon, an interferon receptor or combination thereof, bound by one or more autoantibodies present in the patient sample.

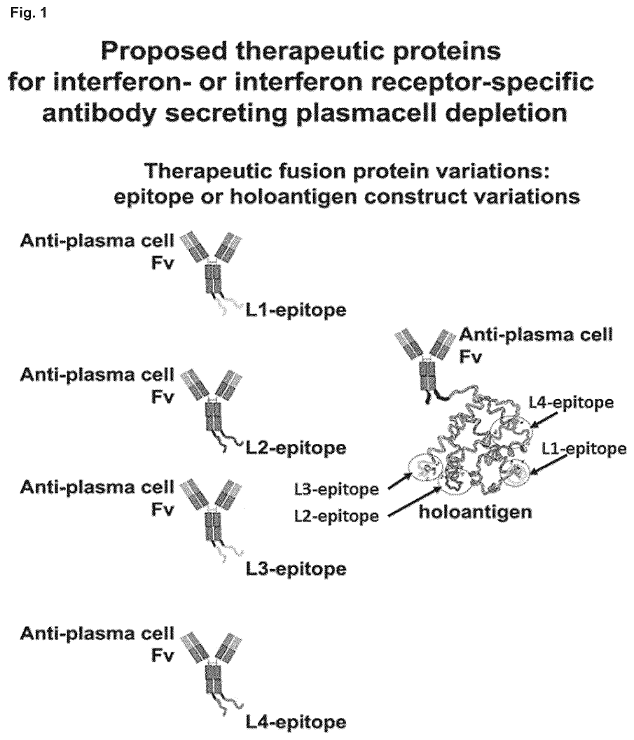

FIG. 2: Schematic depiction showing one embodiment of an in vitro assay according to the invention. Said in vitro assay facilitates the mapping or identification of specific interferon and/or interferon receptor epitopes that are bound by auto-antibodies of a patient. Said autoantibodies are detected in this embodiment in a serum sample of a patient. In variant 1 (v1; embodiment 1) the assay constitutes a binding-inhibition assay which comprises the use of a three-dimensional epitope or even a protein or protein fragment comprising several three-dimensional epitopes. Antibodies with a known binding specificity/affinity to specific epitopes (e.g., of interferons and/or interferon receptors, e.g., such epitopes as exemplary indicated (underlined or underlined/italic) in Table 1) can be labeled with different dyes or fluorophores, such that autoantibodies present in the serum sample are identified, if they compete and prevent the binding of a certain, labelled antibody with a known specificity for an epitope (competition assay). In variant 2 (v2; embodiment 2) epitopes of interferons and/or interferon receptors are immobilized as two-dimensional epitopes on a matrix or surface, e.g., in form of a peptide array, which is used in a similar fashion as in v1. In brief, a competition assay may be performed with said immobilized two-dimensional epitopes between autoantibodies present in a patient sample and antibodies with a known binding specificity/affinity to specific epitopes (e.g., of interferons and/or interferon receptors) that are labeled with different dyes or fluorophores.

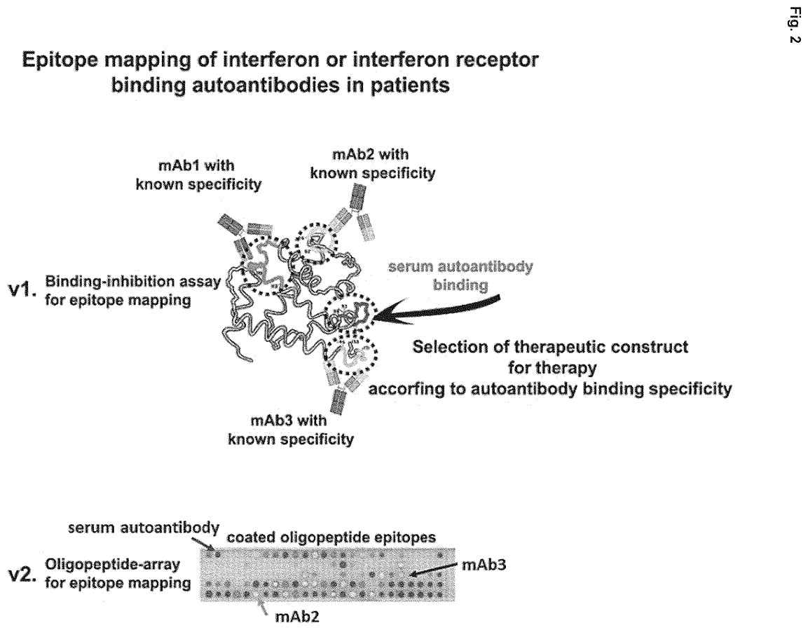

FIG. 3: The Figure shows the results of Example 1, which exemplifies the generation of a bi-specific agent (conjugate) according to the invention. Shown here is a validation experiment wherein the bi-specific agent (conjugate) can be successfully detected on the surface of mouse hybridoma cells expressing CD138. The results evidence a successful conjugate construction. Therein a conjugate comprising an anchor region, namely an anti-mouse CD138 antibody with LALAPA-mutated human IgG1 Fc, was conjugated to a target antigen, namely a recombinant human IFNa2b using SMCC-based chemical coupling.

FIG. 4: The Figure shows the results of Example 2, which exemplifies the detection of anit-IFNa-specific auto-antibodies in the serum of a subject using bi-specific agents (conjugates) according to the invention. Shown here is the successful detection of long-lived antigen-specific plasma cells in bone marrow of antigen-immunized mice using the present bi-specific agents. In the example of Balb/c mice were either immunized with ovabumin (OVA) as control or with human interferon IFNa2b (INFa). (A) 4 month after immunization of the mice, anti-human IFNa2b antibodies were detected in the serum of immunized mice using ELISA. Y-axis: OD 450=Optical density (OD) at 450 nm. X-axis: analyzed immunoglobulins. (B) Anti-human interferon IFNa2b plasma cells were detected in the bone marrow of immunized mice by specific antibody-staining (APC or FITC) and flowcytometry (gating is shown for APC-anti-INFa antibody staining; top left) and OVA (gating is shown for FITC-anti OVA antibody staining; bottom right).

FIG. 5: The Figure shows the results of Example 3, which exemplifies the therapeutic application of an embodiment of the present bi-specific agent (conjugate) in the selective targeting/depletion of autoreactive (here anti-human IFNa2b) plasma cells in a subject. The experiment visualizes the efficacy and specificity of a bi-specific agent (conjugate) according to the invention and the potential for therapeutic usage in human subjects, e.g., in cases where the depletion of human IFNa2b-specific autoreactive plasma cells would be beneficial. In brief: After immunization with ovabumin (OVA) and human interferon IFNa2b, the Balb/c mice were treated with an embodiments of the present bi-specific agent (conjugate) at a concentration of 150 μg/mouse. 2 days after bi-specific agent (conjugate) administration (treatment) anti-OVA and anti-human IFNa2b plasma cells were detected in the bone marrow of immunized mice using (A) flowcytometry and (B) Eilspot (enzyme-linked immunosorbent spot; assay for quantitative measurement of cytokine secretion frequency in single cells).

### EXAMPLES

The invention is further described by the following examples. These are not intended to limit the scope of the invention but represent preferred embodiments of aspects of the invention provided for greater illustration of the invention described herein.

### Example 1

Preparation and functional validation of exemplary conjugates according to the invention targeting human IFNa2b specific mouse plasma cells, wherein the anchor region was conjugated to the target antigen (here human IFNa2b).

### Bi-Specific Agent

Anchor region: anti-mouse CD138 antibody with LALAPA-mutated human IgG1 Fc (purchased from Miltenyi Biotech, Germany).

Target epitope: recombinant human IFNa2b (purchased from Miltenyi Biotec: Ref.: 130-108-967).

Conjugation-type: SMCC-chemical coupling (succinimidyl 4-(N-maleimidomethyl)cyclohexane-1-carboxylate, Sigma-Aldrich; 573114) was used according to standard laboratory protocols.

Bi-specific agent (conjugate): Target antigen conjugated to anchor region.

### Experimental Detection of Successful Generation of Bi-Specific Agents

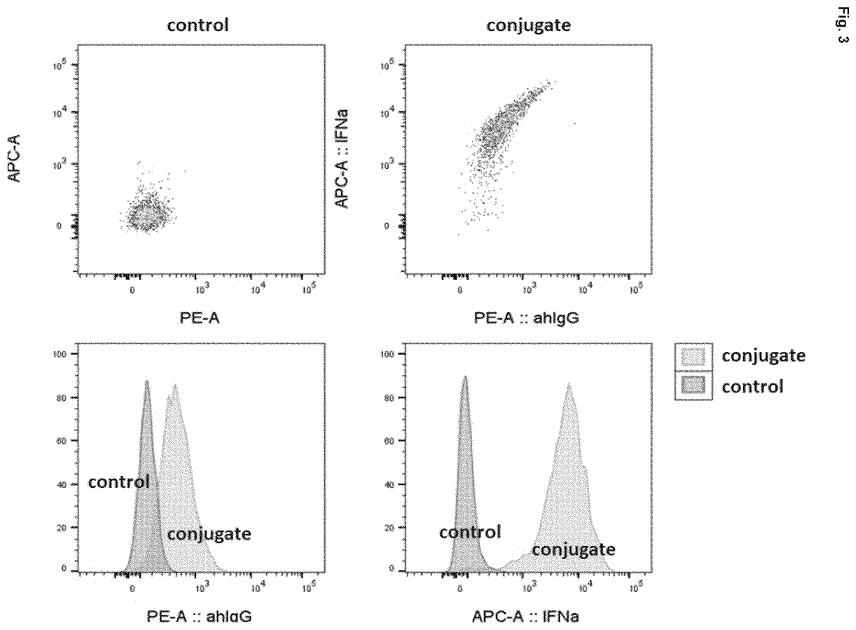

Mouse hybridoma cells expressing CD138 were incubated with the bi-specific agent (conjugate) according to the invention. Subsequently the anchor region of the bi-specific agent (conjugate) was detected using flowcytometry and an anti-human IgG-PE staining. The target epitope was detected using an anti-human IFNa-APC staining. The flowcytometry data shown in FIG. 3 evidences that both anchor region and the target epitope of the conjugate can be detected on the cell surface as a result of successful bi-specific agent (conjugate) construction (coupling).

The present experiment exemplifies the generation of a bi-specific agent according to the invention and shows that a bi-specific agent, comprising an anchor region comprising an anti-mouse CD138 antibody with LALAPA-mutated human IgG1 Fc and a recombinant human IFNa2b, can be successfully detected on the surface of mouse hybridoma cells expressing CD138.

### Example 2

In the present example the specific detection of anti-IFNa-specific auto-antibodies in the serum of human subjects was analyzed by detecting long-lived antigen-specific plasma cells in the bone marrow of antigen-immunized mice.

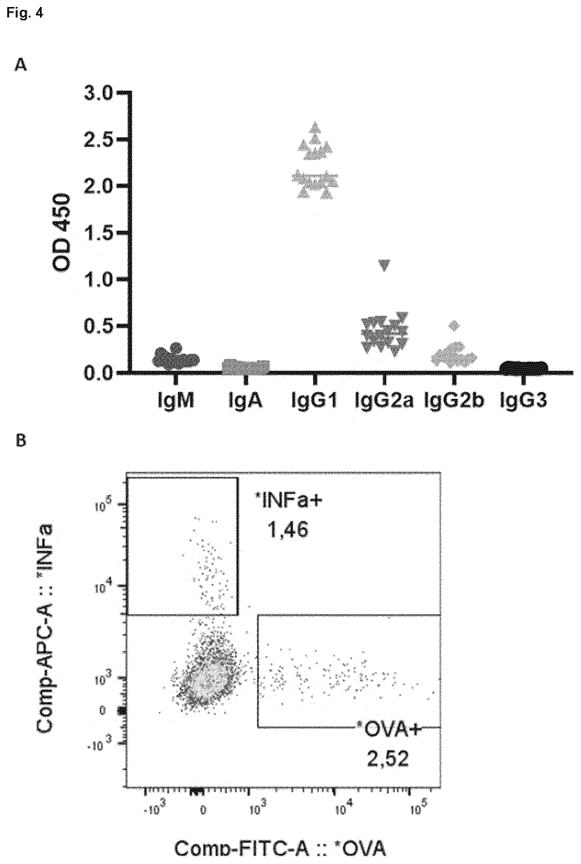

First, Balb/c mice were immunized with ovabumin (OVA) or human interferon IFNa2b. Four month after immunization of the mice anti-human IFNa2b antibodies were detected in the serum of the mice using ELISA (see FIG. 4A). Anti-human IFNa2b plasma cells were detected in the bone marrow of immunized mice using flowcytometry (see FIG. 4B; specific antibody-staining (APC or FITC) with gating for APC-anti-INFa antibody staining and/or FITC-anti OVA antibody staining). The results show that after immunization the mice developed both anti-human IFNa2b antibodies (detected in serum) and long-lived anti-human IFNa2b-specific plasma cells, which could be detected in the bone marrow of the mice. These results indicate that serum samples of humans may also be tested by ELISA for anti-IFNa2b autoantibodies using the bi-specific agent according to the invention, indicating susceptibility for infectious diseases or irresponsiveness to recombinant IFNa2b-therapy. The successful detection of long-lived antigen-specific plasma cells in bone marrow of antigen-immunized mice using the bi-specific agent (conjugate) according to the invention is shown in FIG. 4.

### Example 3

In the present example the selective depletion of anti-human IFNa2b long lived plasma cells was analyzed in vivo, wherein an embodiments of the present bi-specific agent was able to diminish human auto-reactive plasma cells.

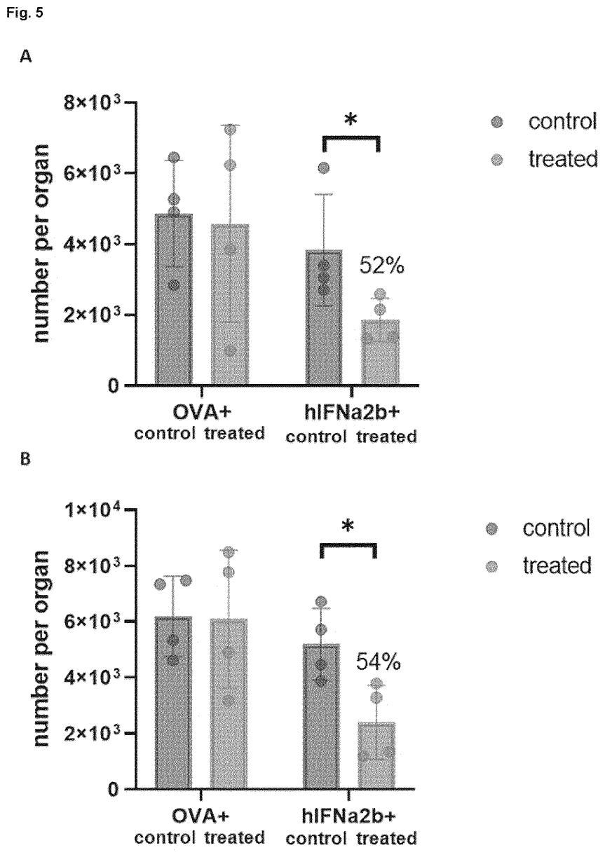

First Balb/c mice were immunized with ovabumin (OVA) or human interferon IFNa2b. Subsequently the mice were treated with a bi-specific agent according to the invention at a concentration of 150 μg per mouse. Two days after administration of the bi-specific agent (treatment of the mice) anti-OVA and anti-human IFNa2b plasma cells were detected in the bone marrow of the immunized mice using flowcytometry (FIG. 5A) and Eilspot (enzyme-linked immunosorbent spot; assay for quantitative measurement of cytokine secretion frequency in single cells) (FIG. 5B). The present example shows an example of the therapeutic application of the present bi-specific agent for selective targeting/depletion of autoreactive (here anti-human IFNa2b) plasma cells in vivo. The experiment visualizes the efficacy and specificity of a bi-specific agent according to the invention and the potential for therapeutic usage in human subjects, e.g., in cases where the depletion of human IFNa2b-specific autoreactive plasma cells would be beneficial.

1-16. (canceled)

17. A bi-specific agent, comprising:
an anchor region capable of binding a plasma cell, and
a binding region associated with the anchor region,
wherein the binding region comprises an epitope of an interferon, an interferon receptor or combination thereof, capable of binding one or more autoantibodies.

18. The bi-specific agent according to claim 17, wherein the autoantibody that binds the epitope of an interferon, an interferon receptor or a combination thereof, is presented on and/or released from a plasma cell (in a patient).

19. The bi-specific agent according to claim 17, wherein the anchor region comprises a monoclonal antibody or antigen-binding fragment thereof that binds or is capable of binding a plasma cell.

20. The bi-specific agent according to claim 17, wherein the anchor region is capable of specifically binding to a plasma cell surface antigen e.g. CD138, CD38, BCMA (TNFRSF17), TACI, CD98, SLAM7 (CD319), FCRL5, CD63, CD74, CD59 or CD27, or combination thereof.

21. The bi-specific agent according to claim 17, wherein the binding region comprises or consists of an amino acid sequence according to SEQ ID NO 1-75 or fragment or variant thereof or underlined sequence from Table 1 or fragment or variant thereof.

22. The bi-specific agent according to claim 17, wherein the anchor region is conjugated to the binding region by a covalent or non-covalent conjugation, bond and/or linker, or the anchor region and binding region are part of the same polypeptide.

23. A method of treating an immunodeficiency in a patient comprising administering to a patient in need thereof, a therapeutically effective amount of the bi-specific agent according to claim 17
wherein the patient is at least 60 years of age or older, and
wherein the immunodeficiency in the patient is associated with the presence of one or more autoantibodies that bind an epitope of an interferon, an interferon receptor or combination thereof, and/or
wherein the patient is or has been treated with recombinant human interferon for another reason, and a presence of one or more autoantibodies that bind an epitope of an interferon is developed as a consequence or side effect of said interferon treatment in the patient.

24. The method according to claim 23, wherein the interferon epitope of the binding region of the bi-specific agent is bound by one or more autoantibodies identified in the patient using an in vitro assay.

25. The method according to claim 24, wherein the in vitro assay comprises:
bringing a sample of the patient comprising antibodies into contact with at least one epitope of an interferon, an interferon receptor or a combination thereof, and
determining the presence of autoantibodies from the patient sample binding to said epitope.

26. The method according to claim 23, wherein the treatment comprises cytotoxicity against plasma cells producing autoantibodies that bind an interferon, an interferon receptor or a combination thereof.

27. An in vitro assay for identifying autoantibodies that bind an epitope of an interferon, an interferon receptor or combination thereof, comprising:
bringing into contact a sample of a patient comprising antibodies with at least one epitope of an interferon, an interferon receptor or combination thereof, and
determining the presence of autoantibodies from the patient sample binding to said epitope.

28. The in vitro assay according to claim 27, comprising bringing into contact the sample of a patient with multiple epitopes of an interferon, an interferon receptor or combination thereof, and determining the epitope bound by said autoantibodies.

29. The in vitro assay according to claim 28, comprising additionally providing a pharmaceutical composition comprising a bi-specific agent, wherein the bi-specific agent comprises:
an anchor region capable of binding a plasma cell, and
a binding region associated with the anchor region,
wherein the binding region comprises an epitope of an interferon, an interferon receptor or combination thereof, capable of binding one or more autoantibodies, and
wherein agent comprises, as the binding region, an epitope of an interferon, an interferon receptor or combination thereof, that was determined to be bound by said autoantibodies.

30. A nucleic acid molecule encoding the bi-specific agent according to claim 17.

31. A method for the in vivo elimination of a plasma cell secreting an autoantibody that binds an epitope of an interferon, an interferon receptor or a combination thereof, the method comprising:
contacting a sample from a patient comprising a plasma cell secreting at least one autoantibody that binds an epitope of an interferon, an interferon receptor or a combination thereof, with a bi-specific agent or a nucleic acid;
wherein the bi-specific agent comprises:
an anchor region capable of binding a plasma cell, and
a binding region associated with the anchor region,
wherein the binding region comprises an epitope of an interferon, an interferon receptor or combination thereof, capable of binding one or more autoantibodies, and
wherein the nucleic acid comprises a nucleic acid molecule encoding the bi-specific agent.

32. A kit for performing the in vitro assay according to claim 27, comprising a bi-specific agent wherein the bi-specific agent comprises:
an anchor region capable of binding a plasma cell, and
a binding region associated with the anchor region,
wherein the binding region comprises an epitope of an interferon, an interferon receptor or combination thereof, capable of binding one or more autoantibodies.

33. The bi-specific agent according to claim 17, wherein the anchor region comprises a monoclonal antibody or antigen-binding fragment thereof that specifically binds or is capable of binding a plasma cell marker presented on the surface of a plasma cell.

34. The method according to claim 23, wherein the interferon treatment has become ineffective in the patient.

35. The method according to claim 25, wherein the in vitro assay comprises:
a competition binding assay using one or more additional antibodies that bind to known epitopes of an interferon, an interferon receptor or a combination thereof,
or
a binding assay using an oligopeptide array comprising multiple epitopes of an interferon, an interferon receptor or a combination thereof.

36. The method according to claim 26, wherein the treatment comprises antibody-dependent cellular cytotoxicity (ADCC) or complement dependent cytotoxicity (CDC) against plasma cells producing autoantibodies that bind an interferon, an interferon receptor or a combination thereof.


SEQUENCE LISTING (75 sequences)

SEQ ID NO: 1 - protein, 189 aa, synthetic construct
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 2 - protein, 188 aa, synthetic construct
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 3 - protein, 189 aa, synthetic construct
MALSFSLLMAVLVLSYKSICSLGCDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 4 - protein, 189 aa, synthetic construct
MALPFVLLMALVVLNCKSICSLGCDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 5 - protein, 190 aa, synthetic construct
TMALPFALLMALVVLSCKSSCSLDCDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 6 - protein, 189 aa, synthetic construct
MALPFALLMALVVLSCKSSCSLDCDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 7 - protein, 189 aa, synthetic construct
MARSFSLLMVVLVLSYKSICSLGCDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLMEKKYSPCAWEVVRAEIMRSFSFSTNLKKGLRRKD

SEQ ID NO: 8 - protein, 189 aa, synthetic construct
MALTFYLLVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLESCVMQEVGVIESPLMYEDSILAVRKYFQRITLYLTEKKYSSCAWEVVRAEIMRSFSLSINLQKRLKSKE

SEQ ID NO: 9 - protein, 189 aa, synthetic construct
MALSFSLLMAVLVLSYKSICSLGCDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 10 - protein, 189 aa, synthetic construct
MASPFALLMALVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 11 - protein, 190 aa, synthetic construct
MMASPFALLMALVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 12 - protein, 189 aa, synthetic construct
MALPFALMMALVVLSCKSSCSLGCNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSLSFSTNLQKRLRRKD

SEQ ID NO: 13 - protein, 189 aa, synthetic construct
MALSFSLLMAVLVLSYKSICSLGCDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKD

SEQ ID NO: 14 - protein, 189 aa, synthetic construct
MALSFSLLMAVLVLSYKSICSLGCDLPQTHSLGNRRALILLAQMGRISPFSCLKDRHDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 15 - protein, 189 aa, synthetic construct
MALSFSLLMAVLVLSYKSICSLGCDLPQTHSLGNRRALILLAQMGRISPFSCLKDRHDFGFPQEEFDGNQFQKAQAISVLHEMIQQTFNLFSTKDSSATWEQSLLEKFSTELNQQLNDLEACVIQEVGVEETPLMNVDSILAVKKYFQRITLYLTEKKYSPCAWEVVRAEIMRSFSLSKIFQERLRRKE

SEQ ID NO: 16 - protein, 187 aa, synthetic construct
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 17 - protein, 212 aa, synthetic construct
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 18 - protein, 189 aa, synthetic construct
MNSFSTIPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 19 - protein, 122 aa, synthetic construct
MCESSKEALAENNLNLPKMAEKDGCFQSGFNEETCLVKIITGLLEFEVYLEYLQNRFESSEEQARAVQMSTKVLIQFLQKKVGVSSFPQLGVGEDRLKDSVLDNSGMQCHFQKRRLHVNKRV

SEQ ID NO: 20 - protein, 212 aa, synthetic construct; identical to SEQ ID NO: 17

SEQ ID NO: 21 - protein, 198 aa, synthetic construct
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKVGVSSFPQLGVGEDRLKDSVLDNSGMQCHFQKRRLHVNKRV

SEQ ID NO: 22 - protein, 136 aa, synthetic construct
MCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 23 - protein, 152 aa, synthetic construct
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQ

SEQ ID NO: 24 - protein, 266 aa, synthetic construct
MNSFSTSKCRKSLALELPAAVEPCVREGCVAQGGLAGGQQQRQAPSCAVSSPLRSLPSGTGAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 25 - protein, 208 aa, synthetic construct
MIIKHFFGTVLVLLASTTIFSLDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 26 - protein, 207 aa, synthetic construct
MSTKPDMIQKCLWLEILMGIFIAGTLSLDCNLLNVHLRRVTWQNLRHLSSMSNSFPVECLRENIAFELPQEFLQYTQPMKRDIKKAFYEMSLQAFNIFSQHTFKYWKERHLKQIQIGLDQQAEYLNQCLEEDKNENEDMKEMKENEMKPSEARVPQLSSLELRRYFHRIDNFLKEKKYSDCAWEIVRVEIRRCLYYFYKFTALFRRK

SEQ ID NO: 27 - protein, 195 aa, synthetic construct
MALLFPLLAALVMTSYSPVGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 28 - protein, 166 aa, synthetic construct
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 29 - protein, 200 aa, synthetic construct
MAAAWTVVLVTLVLGLAVAGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 30 - protein, 200 aa, synthetic construct
MKLDMTGDCTPVLVLMAAVLTVTGAVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 31 - protein, 195 aa, synthetic construct
MTGDCMPVLVLMAAVLTVTGAVPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLC

SEQ ID NO: 32 - protein, 200 aa, synthetic construct
MKLDMTGDCMPVLVLMAAVLTVTGAVPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 33 - protein, 179 aa, synthetic construct
MRPSVWAAVAAGLWVLCTVIAEGPPALPALALPLAGAPDAGGCQGAEGPLRGRGAELGAAQLLLPPQEGSSAAIVLRSAPPRGPGHRGRPGSAQRPAPLGAAPRRRPDPGAAGGRGEGCGGLPAGTARLLQEGPRGPEEASQTPESGLASVPQSQRGLQPPAPAHVGAPAGCTLWALPL

SEQ ID NO: 34 - protein, 107 aa, synthetic construct
MRPSVWAAVAAGLWVLCTVIAEGPPALPALALPLAGAPDAGGCQGAEGPLPAGTARLLQEGPRGPEEASQTPESGLASVPQSQRGLQPPAPAHVGAPAGCTLWALPL

SEQ ID NO: 35 - protein, 131 aa, synthetic construct
MRPSVWAAVAAGLWVLCTVIAEGPPALPALALPLAGAPDAGGCQGAEGPLRGRGAELGAAQLLLPPQEGSSAAIAAGTARLLQEGPRGPEEASQTPESGLASVPQSQRGLQPPAPAHVGAPAGCTLWALPL

SEQ ID NO: 36 - protein, 557 aa, synthetic construct
MMVVLLGATTLVLVAVAPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSKIWLIVGICIALFALPFVIYAAKVFLRCINYVFFPSLKPSSSIDEYFSEQPLKNLLLSTSEEQIEKCFIIENISTIATVEETNQTDEDHKKYSSQTSQDSGNYSNEDESESKTSEELQQDFV

SEQ ID NO: 37 - protein, 458 aa, synthetic construct
MMVVLLGATTLVLVAVAPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSKIWLIVGICIALFALPFVIYAAKVFLRCINYVFFPSLKPSSSIDEYFSEQPLKNLLLSTSEEQIEKCFIIENISTIATVEETNQTDEDHKKYSSQTSQDSGNYSNEDESESKTSEELQQDFV

SEQ ID NO: 38 - protein, 488 aa, synthetic construct
MDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSKIWLIVGICIALFALPFVIYAAKVFLRCINYVFFPSLKPSSSIDEYFSEQPLKNLLLSTSEEQIEKCFIIENISTIATVEETNQTDEDHKKYSSQTSQDSGNYSNEDESESKTSEELQQDFV

SEQ ID NO: 39 - protein, 270 aa, synthetic construct
MMVVLLGATTLVLVAVAPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHLYFRRPR

SEQ ID NO: 40 - protein, 236 aa, synthetic construct
MMVVLLGATTLVLVAVAPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVCILGGRGRWIT

SEQ ID NO: 41 - protein, 128 aa, synthetic construct
MMVVLLGATTLVLVAVAPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKGKD

SEQ ID NO: 42 - protein, 488 aa, synthetic construct; identical to SEQ ID NO: 38

SEQ ID NO: 43 - protein, 303 aa, synthetic construct
MTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSKIWLIVGICIALFALPFVIYAAKVFLRCINYVFFPSLKPSSSIDEYFSEQPLKNLLLSTSEEQIEKCFIIENISTIATVEETNQTDEDHKKYSSQTSQDSGNYSNEDESESKTSEELQQDFV

SEQ ID NO: 44 - protein, 515 aa, synthetic construct [sequence omitted]

SEQ ID NO: 45 - protein, 239 aa, synthetic construct
MLLSQNAFIFRSLNLVLMVYISLVFGISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESEFS

SEQ ID NO: 46 - protein, 331 aa, synthetic construct
MLLSQNAFIFRSLNLVLMVYISLVFGISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKIGGIITVFLIALVLTSTIVTLKWIGYICLRNSLPKVLRQGLAKGWNAVAIHRCSHNALQSETPELKQSSCLSFPSSWDYKRASLCPSD

SEQ ID NO: 47 - protein, 331 aa, synthetic construct; identical to SEQ ID NO: 46

SEQ ID NO: 48 - protein, 167 aa, synthetic construct
MSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESEFS

SEQ ID NO: 49 - protein, 229 aa, synthetic construct
MSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKIGGIITVFLIALVLTSTIVTLKWIGYICLRNSLPKVLNFHNFLAWPFPNLPPLEAMDM

SEQ ID NO: 50 - protein, 204 aa, synthetic construct
MPRIPGICHCKICKSQEKTLKIAKMLLSQNAFIFRSLNLVLMVYISLVFGISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKK

SEQ ID NO: 51 - protein, 515 aa, synthetic construct [sequence omitted]

SEQ ID NO: 52 - protein, 489 aa, synthetic construct
MALLFLLPLVMQGVSRAEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 53 - protein, 479 aa, synthetic construct
MLLKSPENSLLQFQFKYGVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 54 - protein, 455 aa, synthetic construct
MALLFLLPLVMQGVSRAEMGTADLGPSSVPTPTNVTIESYNMNPIVYWEYQIMPQVPVFTVEVKNYGVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 55 - protein, 470 aa, synthetic construct
MCGSIVQSLVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 56 - protein, 448 aa, synthetic construct
MNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 57 - protein, 470 aa, synthetic construct; identical to SEQ ID NO: 55

SEQ ID NO: 58 - protein, 448 aa, synthetic construct; identical to SEQ ID NO: 56

SEQ ID NO: 59 - protein, 479 aa, synthetic construct; identical to SEQ ID NO: 53

SEQ ID NO: 60 - protein, 479 aa, synthetic construct; identical to SEQ ID NO: 53

SEQ ID NO: 61 - protein, 448 aa, synthetic construct; identical to SEQ ID NO: 56

SEQ ID NO: 62 - protein, 448 aa, synthetic construct; identical to SEQ ID NO: 56

SEQ ID NO: 63 - protein, 257 aa, synthetic construct
MALLFLLPLVMQGVSRAEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKDLCGKKCYFRDKT

SEQ ID NO: 64 - protein, 435 aa, synthetic construct
MALLFLLPLVMQGVSRAEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 65 - protein, 437 aa, synthetic construct
MASVFRLYSAVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSF

SEQ ID NO: 66 - protein, 168 aa, synthetic construct
MPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCD

SEQ ID NO: 67 - protein, 337 aa, synthetic construct
MRPTLLWSLLLLLGVFAAAAAAPPDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 68 - protein, 356 aa, synthetic construct
MRPTLLWSLLLLLGVFAAAAAAPPAQLTLETYQEWCNDSAATHDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 69 - protein, 258 aa, synthetic construct
MSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 70 - protein, 334 aa, synthetic construct
PKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVETAFLHVGQTGLELLTSSDLPTSASQSAGITVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQVILISVGTFSLLSVLAGACFFLVLKYRGLIKYWFHTPPSIPLQIEEYLKDPTQPILEALDKDSSPKDDVWDSVSIISFPEKEQEDVLQTL

SEQ ID NO: 71 - protein, 520 aa, synthetic construct
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 72 - protein, 244 aa, synthetic construct
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPGLFWTHTPCGNLSAQQTRVRE

SEQ ID NO: 73 - protein, 211 aa, synthetic construct
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKVGSSFPAPRLGPLLHPFLLRFFSPSQPAPAPLLQEVFPVHS

SEQ ID NO: 74 - protein, 185 aa, synthetic construct
MMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALV

SEQ ID NO: 75 - protein, 491 aa, synthetic construct
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR